SEQUENCE LISTING (22 sequences)

SEQ ID NO: 1 - DNA, 70 bp, Artificial
  Substrate oligonucleotide for glycosylase activity
tccccgaaaagtgccacctgacgtcatcgataagcgtcgaggctcttcgatatcctcgctcactgcattc

SEQ ID NO: 2 - DNA, 70 bp, Artificial
  Substrate oligonucleotide for glycosylase activity
gaatgcagtgagcgaggatatcgaagagcctcgacgcttatcgatgacgtcaggtggcacttttcgggga

SEQ ID NO: 3 - DNA, 70 bp, Artificial
  Substrate oligonucleotide for glycosylase activity
tccccgaaaagtgccacctgacgtcatcgataagcntcgaggctcttcgatatcctcgctcactgcattc

SEQ ID NO: 4 - DNA, 70 bp, Artificial; identical to SEQ ID NO: 1
  Substrate oligonucleotide for glycosylase activity

SEQ ID NO: 5 - DNA, 70 bp, Artificial
  Substrate oligonucleotide for glycosylase activity
tccccgaaaagtgccacctgacgtcatcgataagcutcgaggctcttcgatatcctcgctcactgcattc

SEQ ID NO: 6 - DNA, 70 bp, Artificial
  Substrate oligonucleotide for glycosylase activity
gaatgcagtgagcgaggatatcgaagagcctcgaggcttatcgatgacgtcaggtggcacttttcgggga

SEQ ID NO: 7 - DNA, 70 bp, Artificial
  Substrate oligonucleotide for glycosylase activity
gaatgcagtgagcgaggatatcgaagagcctcgaagcttatcgatgacgtcaggtggcacttttcgggga

SEQ ID NO: 8 - DNA, 70 bp, Artificial
  Padlock probe
gcttatcgatgacgttcatatctcacctacgacctcaatgcacatgtttggctcctcgaagagcctcgac

SEQ ID NO: 9 - DNA, 70 bp, Artificial
  Padlock probe
cgcttatcgatgacgttcatatctcacctacgacctcaatgcacatgtttggctcctcgaagagcctcga

SEQ ID NO: 10 - DNA, 70 bp, Artificial
  Padlock probe
ggatatcgaagagccctatatctcacctacgacctcaatgctgctgctgtactacgaatgcagtgagcga

SEQ ID NO: 11 - DNA, 70 bp, Artificial
  Padlock probe
gcttatcgatgacgttgattactgacctacgacctcaatgctgctgctgtactactcgaagagcctcgaa

SEQ ID NO: 12 - DNA, 70 bp, Artificial
  Padlock probe
gcttatcgatgacgttcatatctcacctacgacctcaatgcacatgtttggctcctcgaagagcctcgag

SEQ ID NO: 13 - DNA, 35 bp, Artificial
  Substrate oligonucleotide for repair
tccccgaaaagtgccacctgacgtcatcgataagc

SEQ ID NO: 14 - DNA, 35 bp, Artificial
  Substrate oligonucleotide for repair
gtcgaggctcttcgatatcctcgctcactgcattc

SEQ ID NO: 15 - DNA, 34 bp, Artificial
  Substrate oligonucleotide for repair
tccccgaaaagtgccacctgacgtcatcgataag

SEQ ID NO: 16 - DNA, 31 bp, Artificial
  Substrate oligonucleotide for repair
tccccgaaaagtgccacctgacgtcatcgat

SEQ ID NO: 17 - DNA, 55 bp, Artificial
  Substrate oligonucleotide for repair
cagatgatagatctgatcaggtcgaggctcttcgatatcctcgctcactgcattc

SEQ ID NO: 18 - DNA, 70 bp, Artificial; identical to SEQ ID NO: 8
  Padlock probe

SEQ ID NO: 19 - DNA, 70 bp, Artificial; identical to SEQ ID NO: 9
  Padlock probe

SEQ ID NO: 20 - DNA, 70 bp, Artificial; identical to SEQ ID NO: 10
  Padlock probe

SEQ ID NO: 21 - DNA, 134 bp, Artificial
  Substrate oligonucleotide for mismatch repair
aacaaataggggttccgcggcattctccccgaaaagtgccacctgacgtcatcgataagcttcgaggctcttcgatatcctcgctcactgacttgctgcgcggatcggttcggctgcggcgagcggtatcagct

SEQ ID NO: 22 - DNA, 134 bp, Artificial
  Substrate oligonucleotide for mismatch repair
agctgataccgctcgccgcagccgaaccgatccgcgcagcaagtcagtgagcgaggatatcgaagagcctcgaggcttatcgatgacgtcaggtggcacttttcggggagaatgccgcggaacccctatttgtt

(19) United States
(12) Patent Application Publication
(10) Pub. No.: US 2011/0027253 A1
(43) Pub. Date: Feb. 3, 2011

(54) PADLOCK PROBE AMPLIFICATION METHODS

(76) Inventors: Jakob Schwalbe Lohmann, Ry (DK); Magnus Stougaard, Hojbjerg (DK); Jørn Erland Koch, Ry (DK)

Correspondence Address: WEINGARTEN, SCHURGIN, GAGNEBIN & LEBOVICI LLP, TEN POST OFFICE SQUARE, BOSTON, MA, 02109, US

(21) Appl. No.: 12/668,517

(22) PCT Filed: Jul. 9, 2008
(86) PCT No.: PCT/DK2008/050174

§ 371 Date: Sep. 8, 2010

Related U.S. Application Data
(60) Provisional application No. 60/949,382, filed on Jul. 12, 2007.
(60) Provisional application No. 61/021,422, filed on Jan. 16, 2008.

(30) Foreign Application Priority Data
Jul. 12, 2007 (DK) PA 2007 01032
Jan. 16, 2008 (DK) PA 2008 00062

(51) Int. Cl. A61K 38/47; C12Q 1/68; C40B 40/06; C40B 30/00; C12M 1/34; C07H 21/00; A61P 43/00

(52) U.S. Cl. 424/94.61; 435/6; 506/16; 506/7; 435/287.2; 536/24.31

(57) ABSTRACT

The present invention relates to an enzyme activity assay using rolling circle 5 amplification for verifying that a sample contains enzyme activity. The enzyme activity assayed is typically involved in processing of mismatched nucleotides and/or damaged nucleotides in a double stranded nucleic acid. The present invention relates to methods for determining the presence of enzyme activities involved in processing double stranded oligonucleotide. Methods are also directed against determining the presence 10 of nucleotide repair enzyme activities involved in the repair of a circular oligonucleotide. The present invention also relates to liquid compositions and solid support both comprising an oligonucleotide probe. Furthermore, the present invention relates to methods for testing the efficacy of a drug, for diagnosing, prognosing, treating a disease by determining the enzyme activity.

All patent and non-patent references cited in this application are hereby incorporated by reference in their entirety.

### FIELD OF INVENTION

The present invention relates to an enzyme activity assay using rolling circle amplification for verifying that a sample contains the enzyme activity in question.

### BACKGROUND OF INVENTION

For DNA modifying enzymes, protein activity detection is primarily performed with techniques using radioactive labeled oligonucleotides. Whilst they are practical for monitoring different cleavage and ligation reactions in solution [1, 2], they are inconvenient because of the radioactive labeling. Another way to measure DNA cleavage and ligation events is by using the Comet assay (also called single-cell gel electrophoresis). In this system, cells are embedded in agarose and lysed.

Subsequently, the nucleoids are electrophorized and the migration of the DNA in the gel-matrix is used as a measurement for how much damage is present in the DNA (reviewed in [3]). By exposing the DNA, before electrophoresis, to either damage causing agents (e.g. UV-light, chemicals, and nucleases) and/or damage repairing agents (e.g. cell extracts and specific repair enzymes) information concerning different repair events can be monitored at an overall level.

Plasmids containing a single mismatch can also be constructed, but these plasmids are time and labor consuming, and the yield is often poor. Heteroduplex plasmids with a mismatch in the blue/white reporter gene β-galactosidase can be used to monitor repair/no repair by transfecting plasmids (following repair) into a mismatch repair-deficient E. coli strain. Subsequently, the ratio of repaired plasmids can be scored. Furthermore, when using human extracts it is an advantage to induce the repair events by positioning a nick 3′ or 5′ to the mismatch in the strand which is going to be repaired.

### SUMMARY OF THE INVENTION

The is a need for innovative methods for the determination of enzyme activities in a biological sample capable of processing e.g. mismatched nucleotides and/or damaged nucleotides in a double stranded oligonucleotide probe.

Mismatches occur when DNA polymerases misinsert nucleotides and fail to proofread the misinserted base. These DNA helix distortions are repaired to minimize introduction of mutations into the genome. The steps in these DNA repair pathways include recognition of the distorted DNA, incision of the DNA by endonucleases on the 5′ or 3′ side of the damage (alternatively a nick is already present in the DNA, omitting the need for endonuclease incision), excision (removal) of nucleotides by exonucleases from the damaged region, and synthesis of a new DNA strand by a DNA polymerase.

A damaged nucleotide opposite a normal nucleotide creates a distortion in the shape of the DNA double helix that is recognized by DNA repair proteins. A DNA helix distortion is also generated when normal, but mismatched nucleotides are generated during DNA replication—for example, if a T nucleotide is paired with C rather than A.

Accordingly, there is a need for assaying a biological sample for enzyme activities capable of processing an unprocessed substrate moiety in a double stranded oligonucleotide probe, such as unprocessed substrate moieties comprising
i) one or more mismatched nucleobase hybridisation event(s) in the double stranded oligonucleotide probe,
ii) a loop formed in the double stranded oligonucleotide probe as a result of the absence of one or more nucleobase hybridisation(s), or
iii) one or more damaged nucleotide(s) in the double stranded oligonucleotide probe.

Additional unprocessed substrate moieties include e.g.
iv) one or more nick(s) in one or more of the strand(s) of the double
v) one or more single stranded nucleotide sequence(s) joined at one or both ends thereof by a double stranded nucleotide sequence, wherein said single stranded sequence(s) create one or more gap structure(s) in the double stranded oligonucleotide probe, and
vi) one or more nick(s) or one or more gap(s), wherein said gap(s) are in the form of a single stranded nucleotide sequence, wherein said nick(s) or gap(s) are joined at one end thereof to a double stranded nucleotide sequence and at the other end thereof to at least one single stranded overhang joined to a double stranded nucleotide sequence of the double stranded nucleotide probe.

Accordingly, in one aspect of the present invention there is provided a method for determining in a biological sample either

a) the presence of one or more enzyme activities involved in processing in a double stranded oligonucleotide probe one or more of
i) one or more mismatched nucleobase hybridisation event(s) in the double stranded oligonucleotide probe, and/or
ii) absence of nucleobase hybridisation(s) resulting in one or more loop formation(s) in the double stranded oligonucleotide probe, and/or
iii) presence of one or more damaged nucleotide(s) in the double stranded oligonucleotide probe, and/or
iv) one or more nick(s) in one or more of the strand(s) of the double stranded nucleotide probe, and/or
v) one or more single stranded nucleotide sequence(s) joined at one or both ends thereof by a double stranded nucleotide sequence, wherein said single stranded sequence(s) create one or more gap structure(s) in
vi) one or more nick(s) or one or more gap(s), wherein said gap(s) are in the form of a single stranded nucleotide sequence, wherein said nick(s) or gap(s) are joined at one end thereof to a double stranded nucleotide sequence and at the other end thereof to at least one single stranded overhang joined to a double stranded nucleotide sequence of the double stranded nucleotide probe,

or

b) the absence of at least one such enzyme activity in said biological sample,

said method comprising the steps of
i) providing a biological sample to be analysed for the presence or absence of at least one enzyme activity,
ii) providing a double stranded oligonucleotide probe comprising an unprocessed substrate moiety capable of being processed by at least one of said one or more enzyme activities,
wherein said unprocessed substrate moiety is selected from the group consisting of
   i) an unprocessed substrate moiety comprising one or more mismatched nucleobase hybridisation event(s) in the double stranded oligonucleotide probe,
   ii) an unprocessed substrate moiety comprising one or more loop formation(s) resulting from absence of nucleobase hybridisation(s) in the double stranded oligonucleotide probe,
   iii) an unprocessed substrate moiety comprising one or more damaged nucleotide(s) in the double stranded oligonucleotide probe,
   iv) an unprocessed substrate moiety comprising one or more nick(s) in one or more of the strand(s) of the double stranded nucleotide probe,
   v) an unprocessed substrate moiety comprising one or more single stranded nucleotide sequence(s) joined at one or both ends thereof by a double stranded nucleotide sequence, wherein said single stranded sequence(s) create one or more gap structure(s) in the double stranded oligonucleotide probe, and
   vi) an unprocessed substrate moiety comprising one or more nick(s) or one or more gap(s), wherein said gap(s) are in the form of a single stranded nucleotide sequence, wherein said nick(s) or gap(s) are joined at one end thereof to a double stranded nucleotide sequence and at the other end thereof to at least one single stranded overhang joined to a double stranded nucleotide sequence of the double stranded nucleotide probe,
wherein said double stranded oligonucleotide probe comprises a single strand of contiguous nucleotides and/or a plurality of single strands of contiguous nucleotides capable of hybridisation to each other,
iii) contacting the biological sample with the double stranded oligonucleotide probe under conditions allowing said one or more enzyme activities, if present in said biological sample, to act on the unprocessed substrate moiety,
wherein said action results in the processing of the unprocessed substrate moiety and the generation of a processed, double stranded oligonucleotide probe,
wherein, when said one or more enzyme activities are not present in said biological sample, no processing of the unprocessed substrate moiety takes place,
iv) separating the individual strands of the double stranded oligonucleotide probe,
v) providing a padlock probe capable of hybridising to an individual strand of the double stranded oligonucleotide probe,
wherein, when the individual strand of the double stranded oligonucleotide probe comprises an unprocessed substrate moiety, the padlock either cannot hybridise to said individual strand comprising an unprocessed substrate moiety, or, when hybridised to said individual strand comprising an unprocessed substrate moiety, the individual strand comprising an unprocessed substrate moiety does not constitute a template for ligation of the nucleotide ends of the padlock probe, in which case the padlock probe cannot be ligated by a ligase and serve as a circular template for rolling circle replication,
wherein, when the individual strand comprises a processed substrate moiety, the padlock probe is hybridised to the said individual strand and ligated by a ligase, thereby providing a circular template for rolling circle replication,
vi) providing a ligase capable of ligating the ends of the padlock probe, thereby generating a circular template for rolling circle replication,
vii) selectively ligating padlock probes hybridised to an individual strand comprising a processed substrate moiety,
wherein said padlock probe ligation results in the formation of a circular oligonucleotide template capable of being amplified by rolling circle replication,
viii) amplifying the circular oligonucleotide template generated in step vii), by using a polymerase capable of performing multiple rounds of rolling circle replication of said circular oligonucleotide template, optionally by contacting said circular oligonucleotide template with a suitable primer, and generating a rolling circle amplification product comprising multiple copies of the circular oligonucleotide template, or
ix) generating no rolling circle amplification product when no padlock probe ligation takes place,
wherein steps viii) and ix) are mutually exclusive,
wherein said amplification product is indicative of the presence in said biological sample of said one or more enzyme activities involved in processing, in a double stranded oligonucleotide probe, one or more of
   i) one or more mismatched nucleobase hybridisation event(s) in the double stranded oligonucleotide probe, and/or
   ii) absence of nucleobase hybridisation(s) resulting in one or more loop formation(s) in the double stranded oligonucleotide probe, and/or
   iii) presence of one or more damaged nucleotide(s) in the double stranded oligonucleotide probe, and/or
   iv) one or more nick(s) in one or more of the strand(s) of the double stranded nucleotide probe, and/or
   v) one or more single stranded nucleotide sequence(s) joined at one or both ends thereof by a double stranded nucleotide sequence, wherein said single stranded sequence(s) create one or more gap structure(s) in the double stranded oligonucleotide probe, and/or
   vi) one or more nick(s) or one or more gap(s), wherein said gap(s) are in the form of a single stranded nucleotide sequence, wherein said nick(s) or gap(s) are joined at one end thereof to a double stranded nucleotide sequence and at the other end thereof to at least one single stranded overhang joined to a double stranded nucleotide sequence of the double stranded nucleotide probe,
and
wherein no rolling circle amplification product is formed in the absence of such an enzyme activity.

It is possible to carry out the above-cited method sequentially for different probes comprising different unprocessed substrate moieties, or alternatively, to carry out the above-cited method in a batch-type reaction, wherein different probes are incubated with the same biological sample material. The probes are preferably coupled to a solid support as disclosed herein elsewhere.

The result of carrying out the above-cited method is a determination of whether or not a biological sample contains at least one enzyme activity, or several enzyme activities, which can be the same or different enzyme activities, depending on the number and nature of probes used, capable of processing the unprocessed substrate moieties of the probe(s) used. The method can be specific and directed to a determination of only one enzyme activity, or one type of enzyme activity, or the method can be directed to a determination, in the same biological sample material, of more than one different enzyme activity, or more than one different type of enzyme activity.

Different unprocessed substrate moieties can be used for analysing enzyme activities contained in the biological sample. The unprocessed substrate moieties can be selected independently from the group consisting of

i) substrate moieties comprising one or more mismatched nucleobase hybridisation event(s) in the double stranded oligonucleotide probe,

ii) substrate moieties comprising a loop formed by the absence of nucleobase hybridisation(s) between one or more nucleotides of different strands of the double stranded oligonucleotide probe,

iii) substrate moieties comprising one or more damaged nucleotide(s) in the double stranded oligonucleotide probe,

iv) substrate moieties comprising one or more nick(s) in one or more of the strand(s) of the double stranded nucleotide probe,

v) substrate moieties comprising one or more single stranded nucleotide sequence(s) joined at one or both ends thereof by a double stranded nucleotide sequence, wherein said single stranded sequence(s) create one or more gap structure(s) in the double stranded oligonucleotide probe, and

vi) substrate moieties comprising one or more nick(s) or one or more gap(s), wherein said gap(s) are in the form of a single stranded nucleotide sequence, wherein said nick(s) or gap(s) are joined at one end thereof to a double stranded nucleotide sequence and at the other end thereof to at least one single stranded overhang joined to a double stranded nucleotide sequence of the double stranded nucleotide probe.

Any combination of the above-cited substrate moieties can in principle occur. Exemplary combinations of probes are cited herein below:

i), ii), iii), iv), v), vi)

i), ii), iii), iv), v)

i), ii), iii), iv), vi)

i), ii), iii), v), vi)

i), ii), iv), v), vi)

i), iii), iv), v), vi)

ii), iii), iv), v), vi)

iii), iv), v), vi)

ii), iv), v), vi)

ii), iii), v), vi)

ii), iii), iv), v)

i), iv), v), vi)

i), iii), v), vi)

i), iii), iv), v)

i), ii), v), vi)

i), ii), iv), vi)

i), ii), iv), v)

i), ii), iii), vi)

i), ii), iii), v)

i), ii), iii), iv)

i), ii), iii)

i), ii), iv)

i), ii), v)

i), ii), vi)

ii), iii), iv)

ii), iii), v)

ii), iii), vi)

iii), iv), v)

iii), iv), vi)

iii), iv), i)

iv), v), vi)

iv), v), i)

iv), v), ii)

v), vi), i)

v), vi), ii)

v), vi), iii)

i), iii), v)

i), iii), vi)

i), ii)

i), iii)

i), iv)

i), v)

i), vi)

ii), iii)

ii), iv)

ii), v)

ii), vi)

iii), iv)

iii), v)

iii), vi)

iv), v)

v), vi)

In the absence in the biological sample of an enzyme activity capable of processing a probe oligonucleotide comprising an unprocessed substrate moiety, no rolling circle replication event will be generated as explained in more detail herein below, and accordingly, no proof of the processing will be provided.

The method employs the steps of providing a biological sample to be analysed for the presence or absence of at least one enzyme activity and providing a double stranded oligonucleotide probe comprising an unprocessed substrate moiety capable of being processed by at least one of said one or more enzyme activities, wherein said double stranded oligonucleotide probe comprises a single strand of contiguous nucleotides and/or a plurality of single strands of contiguous nucleotides capable of hybridisation to each other.

The biological sample is contacted with the double stranded oligonucleotide probe under conditions allowing said one or more enzyme activities, if present in said biological sample, to act on the unprocessed substrate moiety. Provided that an enzyme activity is present in the biological sample which is capable of processing one or more of the provided unprocessed substrate moieties, the enzymatic action results in the processing of the unprocessed substrate moiety and the generation of a processed, double stranded oligonucleotide probe. When said one or more enzyme activities are not present in said biological sample, no processing of the unprocessed substrate moiety takes place.

After the contacting of the oligonucleotide probe and the biological sample, the individual strands of the double stranded oligonucleotide probe are separated or displaced. This can happen by any method known to the skilled person, including by enzymatic digestion of one of the strands, preferably the strand not comprising the unprocessed or processed substrate moiety, as it may be.

Once the individual strands of the double stranded oligonucleotide probe have been separated from each other, a padlock probe capable of hybridising to an individual strand of the double stranded oligonucleotide probe is provided. The padlock probe preferably hybridises to a strand comprising a processed substrate moiety. When the individual strand of the double stranded oligonucleotide probe comprises an unprocessed substrate moiety, the padlock probe either cannot hybridise to said individual strand comprising an unprocessed substrate moiety, or, when hybridised to said individual strand comprising an unprocessed substrate moiety, the individual strand comprising an unprocessed substrate moiety does not constitute a template for ligation of the nucleotide ends of the padlock probe, in which case the padlock probe cannot be ligated by a ligase and serve as a circular template for rolling circle replication.

When the individual strand comprises a processed substrate moiety, the padlock probe is hybridised to the said individual strand and ligated by a ligase, thereby providing a circular template for rolling circle replication,

In the next step, a ligase capable of ligating the ends of the padlock probe is provided. Once ligated the padlock probe is in the form of a circular template for rolling circle replication.

Two scenarios are possible at this stage of the method. Either, a padlock probe hybridised to an individual strand comprising a processed substrate moiety is ligated, wherein said padlock probe ligation results in the formation of a circular oligonucleotide template capable of being amplified by rolling circle replication. Following padlock probe circularisation and formation of a circular oligonucleotide template, the circular oligonucleotide template is amplified by using a polymerase capable of performing multiple rounds of rolling circle replication of said circular oligonucleotide template, optionally by contacting said circular oligonucleotide template with a suitable primer, and generating a rolling circle amplification product comprising multiple copies of the circular oligonucleotide template. Preferably, the strand to which the padlock probe has been hybridized and ligated is used as the primer.

Alternatively, no rolling circle amplification product is generated as no padlock probe ligation takes place—either because no padlock probe hybridisation to the individual strand of the double stranded oligonucleotide probe takes place, or because the padlock probe is hybridised to an individual strand comprising an unprocessed substrate moiety—in which case no substrate for ligation of the padlock probe is provided.

The formation of a rolling circle amplification product is indicative of the presence in said biological sample of said one or more enzyme activities involved in processing an unprocessed substrate moiety in the probe oligonucleotide employed for the assay. No rolling circle amplification product is formed in the absence of such an enzyme activity.

It is another object of the present invention to provide a method for determining in a biological sample either
a) the presence of one or more nucleotide repair enzyme activities involved in repairing a damaged nucleotide in a circular oligonucleotide probe, or
b) the absence of such a nucleotide repair enzyme activity,
said method comprising the steps of
   i) providing a biological sample to be analysed for the presence or absence of a nucleotide repair enzyme activity involved in repairing a damaged nucleotide,
   ii) providing an oligonucleotide probe comprising one or more strands,
   wherein at least one of said one or more strands is in the form of a circular oligonucleotide comprising a damaged nucleotide capable of being repaired by the nucleotide repair enzyme activity,
   wherein, preferably, said oligonucleotide probe is selected from the group consisting of a circular, self-templated oligonucleotide probe comprising a single strand of contiguous nucleotides capable of hybridising to a primer for priming rolling circle amplification of said circular, self-templated oligonucleotide probe and a circular oligonucleotide probe comprising a plurality of single strands of contiguous nucleotides capable of hybridisation to each other, wherein at least one of said single strands is in the form of a circular oligonucleotide, wherein at least one of the plurality of single strands is capable of hybridising to the circular oligonucleotide and priming rolling circle amplification of said circular oligonucleotide,
   iii) incubating the biological sample and the circular oligonucleotide probe comprising a damaged nucleotide capable of being repaired by the nucleotide repair enzyme activity under conditions allowing said nucleotide repair enzyme activity, if present in said biological sample, to act on the damaged nucleotide,
   wherein said action results in repairing the damaged nucleotide, and
   wherein no repair of the damaged nucleotide occurs in the absence of said one or more nucleotide repair enzyme activities in said biological sample,
   iv) providing a glycosylase enzyme activity and contacting the glycosylase enzyme activity with the self-templating circular oligonucleotide probe provided in step ii) after said probe has been incubated with the biological sample,
   wherein the glycosylase enzyme activity cleaves the circular oligonucleotide strand having an unrepaired and damaged nucleotide, thereby generating a linear oligonucleotide which cannot be amplified by rolling circle amplification, and
   wherein the glycosylase enzyme activity does not cleave the circular oligonucleotide strand having a repaired nucleotide, wherein said circular oligonucleotide strand can be amplified by rolling circle replication,
   v) priming the circular oligonucleotide strand having been contacted with the glycosylase enzyme activity, optionally by providing a primer, and
   vi) amplifying the circular oligonucleotide strand comprising a repaired nucleotide, when such a circular oligonucleotide strand is formed in step iv), by using a polymerase capable of performing multiple rounds of rolling circle replication of said circular oligonucleotide strand, and generating a rolling circle amplification product comprising multiple copies of the circular oligonucleotide strand, or
   viii) generating no rolling circle amplification product when no circular oligonucleotide strand is formed in step iv) as a result of said glycosylase having excised said damaged nucleotide from said circular oligonucleotide strand and thereby having generated a linear oligonucleotide strand,
   wherein steps vi) and vii) are mutually exclusive,
   wherein said amplification product is indicative of the presence in said biological sample of a nucleotide repair enzyme activity involved in repairing a damaged nucleotide in a circular oligonucleotide strand
   and
   wherein no amplification product is formed in the absence of such an enzyme activity.

The method is specific for the determination in biological samples of nucleotide repair enzyme activities involved in repairing a damaged nucleotide. A damaged nucleotide opposite a normal nucleotide creates a distortion in the shape of the DNA double helix that is recognized by DNA repair proteins. A DNA helix distortion is also generated when normal, but mismatched nucleotides are generated during DNA replication, for example, if a nucleotide T is paired with C rather than A.

In an initial method step there is provided an oligonucleotide probe comprising one or more strands, wherein at least one of said one or more strands is in the form of a circular oligonucleotide comprising a damaged nucleotide capable of being repaired by the nucleotide repair enzyme activity,

The oligonucleotide probe can be a circular, self-templated oligonucleotide probe comprising a single strand of contiguous nucleotides capable of hybridising to a primer for priming rolling circle amplification of said circular, self-templated oligonucleotide probe. The oligonucleotide probe can also be in the form of a circular oligonucleotide probe comprising a plurality of single strands of contiguous nucleotides capable of hybridisation to each other, wherein at least one of said single strands is in the form of a circular oligonucleotide, wherein at least one of the plurality of single strands is capable of hybridising to the circular oligonucleotide and priming rolling circle amplification of said circular oligonucleotide.

The biological sample and the circular oligonucleotide probe comprising a damaged nucleotide capable of being repaired by the nucleotide repair enzyme activity are incubated under conditions allowing said nucleotide repair enzyme activity, if present in said biological sample, to act on the damaged nucleotide, wherein said action results in repairing the damaged nucleotide.

No repair of the damaged nucleotide occurs in the absence of said one or more nucleotide repair enzyme activities in said biological sample.

A glycosylase enzyme activity is provided and contacted with the self-templating circular oligonucleotide probe after said probe has been incubated with the biological sample. The glycosylase enzyme activity cleaves a circular oligonucleotide strand having an unrepaired and damaged nucleotide, thereby generating a linear oligonucleotide which cannot be amplified by rolling circle amplification. The glycosylase enzyme activity does not cleave a circular oligonucleotide strand having a repaired nucleotide, which means that the circular oligonucleotide strand can be amplified by rolling circle replication.

The circular oligonucleotide strand having been contacted with the glycosylase enzyme activity is primed, optionally by providing a primer, and the circular oligonucleotide strand comprising a repaired nucleotide, when such a circular oligonucleotide strand is formed, is amplified by using a polymerase capable of performing multiple rounds of rolling circle replication of said circular oligonucleotide strand, and generating a rolling circle amplification product comprising multiple copies of the circular oligonucleotide strand. No rolling circle amplification product is generated when no circular oligonucleotide strand is formed as a result of said glycosylase having excised said damaged nucleotide from said circular oligonucleotide strand and thereby having generated a linear oligonucleotide strand.

The amplification product is indicative of the presence in said biological sample of a nucleotide repair enzyme activity involved in repairing a damaged nucleotide in a circular oligonucleotide strand. No such amplification product is formed in the absence in the biological sample of such an enzyme activity.

A third aspect of the invention relates to a liquid composition comprising
a) one or more oligonucleotide probes selected from the group consisting of
   i) oligonucleotide probes comprising unprocessed substrate moieties comprising or consisting of one or more nick(s) in one or more single strand(s) of a double stranded nucleotide sequence of said oligonucleotide probe, said one or more nick(s) forming one or more unprocessed substrate moieties of said oligonucleotide probe,
   ii) oligonucleotide probes comprising unprocessed substrate moieties comprising or consisting of one or more single stranded nucleotide sequence(s) joined at one or both ends thereof by a double stranded nucleotide sequence, said single stranded sequence(s) creating one or more gap structure(s) forming one or more unprocessed substrate moieties of said oligonucleotide probe,
   iii) oligonucleotide probes comprising unprocessed substrate moieties comprising or consisting of one or more nick(s) or one or more gap(s), said gap(s) being in the form of a single stranded nucleotide sequence, said nick(s) or gap(s) being joined at one end thereof to a double stranded nucleotide sequence and at the other end thereof to at least one single stranded overhang joined to a double stranded nucleotide sequence of said oligonucleotide probe, wherein said nick(s) or gap(s) in combination with the at least one single stranded overhang forms one or more unprocessed substrate moieties of said oligonucleotide probe;
   iv) oligonucleotide probes comprising unprocessed substrate moieties comprising or consisting of one or more mismatched nucleobase hybridisation event(s) in the double stranded oligonucleotide probe, and/or
   v) oligonucleotide probes comprising unprocessed substrate moieties comprising or consisting of the absence of nucleobase hybridisation(s) resulting in one or more loop formation(s) in the double stranded oligonucleotide probe, and/or
   vi) oligonucleotide probes comprising unprocessed substrate moieties comprising or consisting of one or more damaged nucleotide(s) in the double stranded oligonucleotide probe, and/or
   and
b) a liquid carrier, such as an aqueous solvent, allowing one or more enzymes to process the one or more unprocessed substrate moieties of said one or more oligonucleotide probes.

A fourth aspect pertains to a composition comprising a tissue sample, or a biopsy sample, obtained from an animal, such as a human being, and the liquid composition as disclosed above.

A fifth aspect relates to a solid support coupled to the oligonucleotide probe as defined herein.

Another aspect relates to a solid support comprising a plurality of attachment points for the attachment to the solid support of one or more oligonucleotide probes each comprising one or more unprocessed substrate moieties, wherein an oligonucleotide probe is either directly attached to an attachment point through one strand of the oligonucleotide probe, wherein said strand is capable of initiating rolling circle amplification of a second strand of the oligonucleotide probe, or an oligonucleotide probe is attached to an attachment point through hybridisation of the oligonucleotide probe to a primer oligonucleotide attached to an attachment point, wherein said primer is capable of initiating rolling circle amplification of the oligonucleotide probe, so that individual attachment points are associated with one or more oligonucleotide primers suitable for initiating rolling circle amplification of a circular template generated by enzyme processing of said one or more oligonucleotide probes each comprising one or more unprocessed substrate moieties,
wherein the same or different primers are associated with the same or different attachment points,
wherein the oligonucleotide probes attached to the solid support are selected from the group consisting of
   i) oligonucleotide probes comprising unprocessed substrate moieties comprising or consisting of one or more nick(s) in one or more single strand(s) of a double stranded nucleotide sequence of said oligonucleotide probe, said one or more nick(s) forming one or more unprocessed substrate moieties of said oligonucleotide probe,
   ii) oligonucleotide probes comprising unprocessed substrate moieties comprising or consisting of one or more single stranded nucleotide sequence(s) joined at one or both ends thereof by a double stranded nucleotide sequence, said single stranded sequence(s) creating one or more gap structure(s) forming one or more unprocessed substrate moieties of said oligonucleotide probe,
   iii) oligonucleotide probes comprising unprocessed substrate moieties comprising or consisting of one or more nick(s) or one or more gap(s), said gap(s) being in the form of a single stranded nucleotide sequence, said nick(s) or gap(s) being joined at one end thereof to a double stranded nucleotide sequence and at the other end thereof to at least one single stranded overhang joined to a double stranded nucleotide sequence of said oligonucleotide probe, wherein said nick(s) or gap(s) in combination with the at least one single stranded overhang forms one or more unprocessed substrate moieties of said oligonucleotide probe,
   iv) oligonucleotide probes comprising unprocessed substrate moieties comprising or consisting of one or more mismatched nucleobase hybridisation event(s) in the double stranded oligonucleotide probe,
   v) oligonucleotide probes comprising unprocessed substrate moieties comprising or consisting of the absence of nucleobase hybridisation(s) resulting in one or more loop formation(s) in the double stranded oligonucleotide probe, and
   vi) oligonucleotide probes comprising unprocessed substrate moieties comprising or consisting of one or more damaged nucleotide(s) in the double stranded oligonucleotide probe.

Yet another aspect relates to a microfluidic device comprising one or more reaction compartments for performing one or more rolling circle amplification events of a circular oligonucleotide template and one or more detection compartments for the detection of said rolling circle amplification events performed in said one or more reaction compartments. In embodiments of the present invention the microfluidic device further comprises the solid support as described elsewhere herein.

Yet a further aspect relates to a method for correlating one or more rolling circle amplification event(s) with the activity of one or more enzymes in a sample, said method comprising the steps of performing the method according to the present invention and amplifying by rolling circle amplification the one or more circular templates having been generated as a result of the presence in said sample of said one or more enzyme activities, wherein the detection of said amplification events is done using the solid support as described elsewhere herein or the microfluidic device also described elsewhere herein, wherein a predetermined number of rolling circle amplification events correlate with a predetermined enzyme activity, and wherein the actual number of rolling circle amplification events recorded for a given sample is compared to the number of events correlating with said predetermined enzyme activity, thereby correlating the actual number of rolling circle amplification events with said activity of said one or more enzyme activities present in said sample.

The present invention thus also relates to an aspect which pertains to a method for testing the efficacy of a drug or drug-lead, said method comprising the steps of
i) providing a drug or drug-lead to be tested;
ii) providing a biological sample to be treated with the drug or drug-lead;
iii) performing the correlation method as described elsewhere herein for the biological sample in the absence of drug or drug-lead and determining the activity of one or more enzyme activities involved in circularising a non-circular oligonucleotide probe;
iv) contacting the drug or drug-lead and the biological sample;
v) performing the correlation method as described elsewhere herein for the biological sample in the presence of drug or drug-lead and determining the activity of one or more enzyme activities involved in circularising a non-circular oligonucleotide probe;
vi) comparing the enzyme activities in the biological sample in the presence and absence, respectively, of the drug or drug-lead, wherein said comparison is obtained by comparing the rolling circle amplification events in the presence and absence, respectively, of the drug or drug-lead, and
vii) evaluating the efficacy of the drug or drug-lead based on the comparison performed in step vi).

Furthermore, another aspect relates to a method for diagnosing or prognosing a disease in an individual by determining the activity of one or more enzyme activities involved in circularising a non-circular oligonucleotide probe, said method comprising the steps of
i) obtaining a biological sample from an individual to be tested, said biological sample comprising said one or more enzyme activities to be tested in the diagnostic or prognostic method,
ii) performing on said biological sample the method as described herein and amplifying by rolling circle amplification the one or more circular templates having been generated as a result of the presence in said sample of said one or more enzyme activities being tested for, and optionally detecting said amplification events by using the solid support as described elsewhere herein or the microfluidic device as described elsewhere herein, and
iii) determining the number of rolling circle amplification events and
iv) correlating said number of rolling amplification events with a predetermined enzyme activity corresponding to standard defining a physiologically normal activity of the one or more enzyme activities being tested for in a healthy individual,
wherein the actual number of rolling circle amplification events recorded for a given sample is compared to the number of events correlating with said predetermined enzyme activity, thereby correlating the actual number of rolling circle amplification events with said activity of said one or more enzyme activities present in said sample, and diagnosing or prognosing said individual with said disease, or the likelyhood of developing said disease, based on the enzyme activities determined in said biological sample.

Once a diagnosis is determined as described above the present invention pertains to yet a further aspect, namely a method for treating a disease diagnosed as described above, said method comprising the steps of administering a pharmaceutical composition to said individual having being diagnosed with said disease, wherein said medicament is capable of treating said disease by curing the disease or ameliorating the disease.

Definitions

Biological sample: Any sample comprising biological material, such as an enzymatic activity, a tissue sample and/or a body fluid sample. Any sample comprising an enzymatic activity capable of converting an unprocessed substrate moiety is a biological sample.

Mismatched nucleobase: Mismatches occur when DNA polymerases misinsert nucleotides and fail to proofread the misinserted base. These DNA helix distortions are repaired to minimize introduction of mutations into the genome. The steps in these DNA repair pathways include recognition of the distorted DNA, incision of the DNA by endonucleases on the 5′ or 3′ side of the damage (alternatively a nick is already present in the DNA, omitting the need for endonuclease incision), excision (removal) of nucleotides by exonucleases from the damaged region, and synthesis of a new DNA strand by a DNA polymerase. Correctly matched deoxyribonucleotides include any of the natural deoxyribonucleotides matched (correctly hybridised) to a natural hybridisation partner, i.e. A hybridised to T and C hybridised to G.

Loop formation: Two nucleotide strands can form a loop if internal nucleotides does not base-pair with each other.

Damaged nucleotide: A damaged nucleotide opposite a normal nucleotide creates a distortion in the shape of the DNA double helix that is recognized by DNA repair proteins. A DNA helix distortion is also generated when normal, but mismatched nucleotides are generated during DNA replication—for example, if a T nucleotide is paired with C rather than A.

Unprocessed substrate moiety: An entity capable of being processed by one or more enzyme activities present in a biological sample.

Padlock probe: A padlock probe is a linear oligonucleotide comprising at least two target complementary sequences separated by a linker. The padlock probe can be circularized by ligation in the presence of a correct target sequence. The target sequence thus acts as a hybridisation partner for the padlock probe. Padlock probe hybridisation to a target sequence may not be sufficient to ensure padlock probe ligation. Target sequences in the form of hybridisation partners comprising an unprocessed substrate moiety does not serve as a template for padlock probe ligation. Accordingly, even if a padlock probe hybridises to a target sequence comprising an unprocessed substrate moiety, no padlock probe ligation can result from the hybridisation.

Gap: A double stranded region of DNA wherein one of the strands possesses a free 5′-end and a free 3′-end separated by a gap of one or more nucleotides.

Nick: A double stranded region of DNA wherein one of the strands possesses a free 5′-end and a free 3′-end separated by a gap of zero nucleotides.

Artificial nucleic acid: That being both nucleic acids not found in the nature, e.g. but not limited to, PNA, LNA, iso-dCTP, or iso-dGTP, as well as any modified nucleotide, e.g., but not limited to, biotin coupled nucleotides, fluorophore coupled nucleotides, or radioactive nucleotides.

Closed circular structure: A nucleic acid sequence with a non-ending backbone, e.g., but not limited to, sugar-phosphate in DNA and RNA, or N-(2-aminoethyl)-glycine units linked by peptide bonds in PNA.

Hybridise: Base pairing between two complementary nucleic acid sequences. Hybridisation can be matched or mis-matched.

Intra-molecular structure: Hybridisation of one or more nucleic acid sequence parts in a molecule to one or more nucleic acid sequence parts of the same molecule.

LNA: Locked nucleic acid.

Natural nucleic acids: The nucleotides G, C, A, T, U and I.

Open circular structure: A nucleic acid sequence which is in a circular structure, either aided by an external ligation template or self-templated, with at least one 5′-end and one 3′-end. E.g., but not limited to, sugar-phosphate in DNA and RNA, or N-(2-aminoethyl)-glycine units linked by peptide bonds in PNA.

PNA: Peptide nucleic acid.

Double stranded oligonucleotide probe: One or more nucleic acid sequence(s) composed of natural or artificial, modified or unmodified nucleotides. The probe comprises one or more unprocessed substrate moieties.

Circular template for rolling circle replication: A ligated padlock probe or a self-templated probe comprising a closed, circular sequence of nucleotides, artificial or natural, that a polymerase can use as a template during rolling circle replication.

### BRIEF DESCRIPTION OF THE FIGURES

FIG. 1: Repair of Double Stranded Repair Substrate for Mismatch, Loop, or Damaged Bases without Nick

A double stranded repair substrate coupled to a solid support. The wobble corresponds to a mismatch, a loop, or a damaged base. The wobble can be repaired, by one or more enzymes. To get a 3′-end closer to the repair site a restriction digestion can optionally be introduced. Following repair/no repair, the substrate can be made single stranded by either denaturation or exonuclease digestion. Subsequently a padlock probe can be hybridized and ligated. The ligation step depends on repair of the wobble. Subsequently the ligation event can be visualized by rolling circle DNA synthesis and fluorescence detection.

FIG. 2: 3′ Nick Induced Repair

A double stranded repair substrate coupled to a solid support containing a nick positioned 3′ to the wobble. The wobble corresponds to a mismatch, a loop, or a damaged base. The wobble can be repaired, by one or more enzymes. To get a 3′-end closer to the repair site a restriction digestion can optionally be introduced. Following repair/no repair, the substrate can be made single stranded by either denaturation or exonuclease digestion. Subsequently a padlock probe can be hybridized and ligated. The ligation step depends on repair of the wobble. Subsequently the ligation event can be visualized by rolling circle DNA synthesis and fluorescence detection.

FIG. 3: 5′ Nick Induced Repair

A double stranded repair substrate coupled to a solid support containing a nick positioned 5′ to the wobble. The wobble corresponds to a mismatch, a loop, or a damaged base. The wobble can be repaired, by one or more enzymes. To get a 3′-end closer to the repair site a restriction digestion can optionally be introduced. Following repair/no repair, the substrate can be made single stranded by either denaturation or exonuclease digestion. Subsequently a padlock probe can be hybridized and ligated. The ligation step depends on repair of the wobble. Subsequently the ligation event can be visualized by rolling circle DNA synthesis and fluorescence detection.

FIG. 4: Repair of Gaps

A double stranded repair substrate coupled to a solid support containing a gap. The gap can be repaired, by one or more enzymes. To get a 3′-end closer to the repair site a restriction digestion can optionally be introduced. Following repair/no repair, the substrate can be made single stranded by either denaturation or exonuclease digestion. Subsequently a padlock probe can be hybridized and ligated. The ligation step depends on repair of the wobble. Subsequently the ligation event can be visualized by rolling circle DNA synthesis and fluorescence detection.

FIG. 5: Repair of 5′ Overhangs

A double stranded repair substrate coupled to a solid support containing a 5′ overhang. The 5′ overhang can be repaired, by one or more enzymes. To get a 3′-end closer to the repair site a restriction digestion can optionally be introduced. Following repair/no repair, the substrate can be made single stranded by either denaturation or exonuclease digestion. Subsequently a padlock probe can be hybridized and ligated. The ligation step depends on repair of the wobble. Subsequently the ligation event can be visualized by rolling circle DNA synthesis and fluorescence detection.

FIG. 6: Repair of 3′ Overhangs

A double stranded repair substrate coupled to a solid support containing a 3′ overhang. The 3′ overhang can be repaired, by one or more enzymes. To get a 3′-end closer to the repair site a restriction digestion can optionally be introduced. Following repair/no repair, the substrate can be made single stranded by either denaturation or exonuclease digestion. Subsequently a padlock probe can be hybridized and ligated. The ligation step depends on repair of the wobble. Subsequently the ligation event can be visualized by rolling circle DNA synthesis and fluorescence detection.

FIG. 7: Repair of Damaged Nucleotide using Self-Templating Circular Probes

A circular self-templating probe equipped with a damaged nucleotide. The damaged nucleotide can be repaired by one or more enzymes. Following repair, un-repaired probes can be linearized using a specific glycosylase recognizing the damaged base. A repaired probe will not be recognized by the glycosylase. Subsequently repaired probes can be amplified by rolling circle DNA synthesis and detected by fluorescence detection.

FIG. 8: Repair of Damaged Nucleotide using Circular Probes

A circular probe equipped with a damaged nucleotide. The damaged nucleotide can be repaired by one or more enzymes. Following repair, un-repaired probes can be linearized using a specific glycosylase recognizing the damaged base. A repaired probe will not be recognized by the glycosylase. Subsequently repaired probes can be amplified by rolling circle DNA synthesis and detected by fluorescence detection.

FIG. 9: Purines and Pyrimidines

Structure of the standard purines and pyrimidines

FIG. 10: List of the Major Oxidative Damages to Bases

FIG. 11: Target Sites for Intracellular DNA Decay.

FIG. 12: Enzymes Known to be Involved in DNA Repair

FIG. 13: Detection of the Repair of an Abasic Site in a Double Stranded Substrate by a Cell Extract.

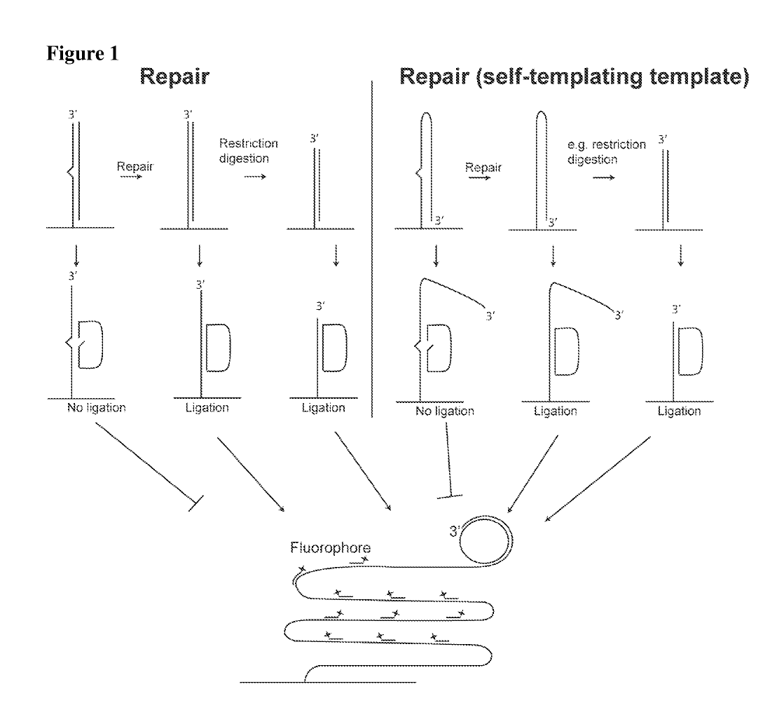

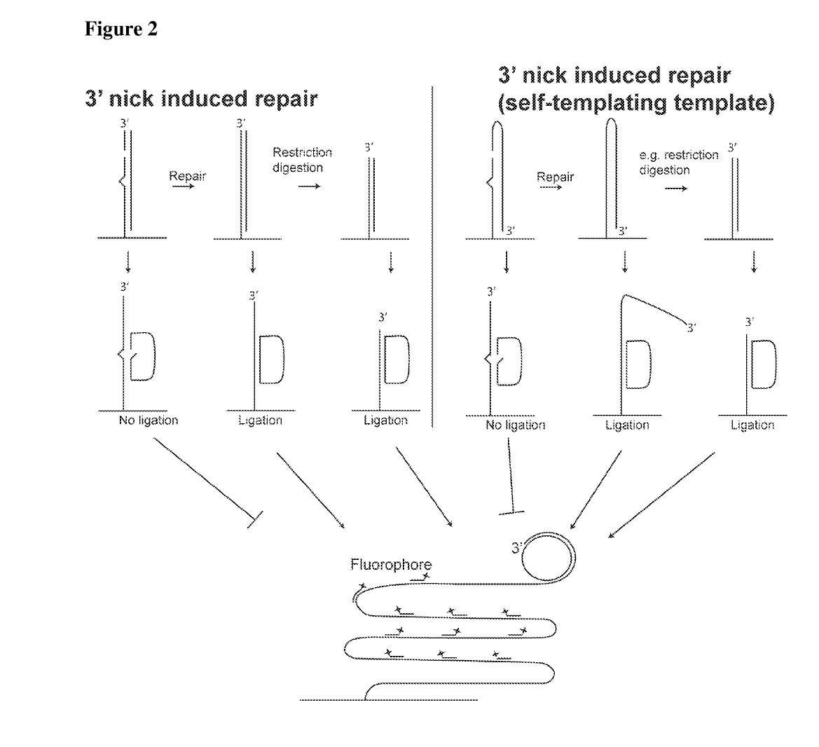

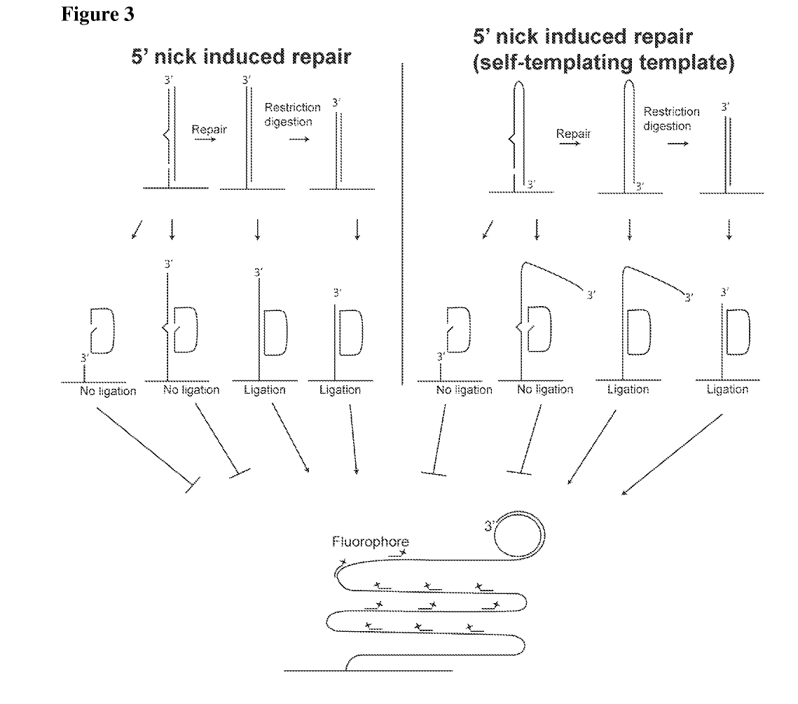

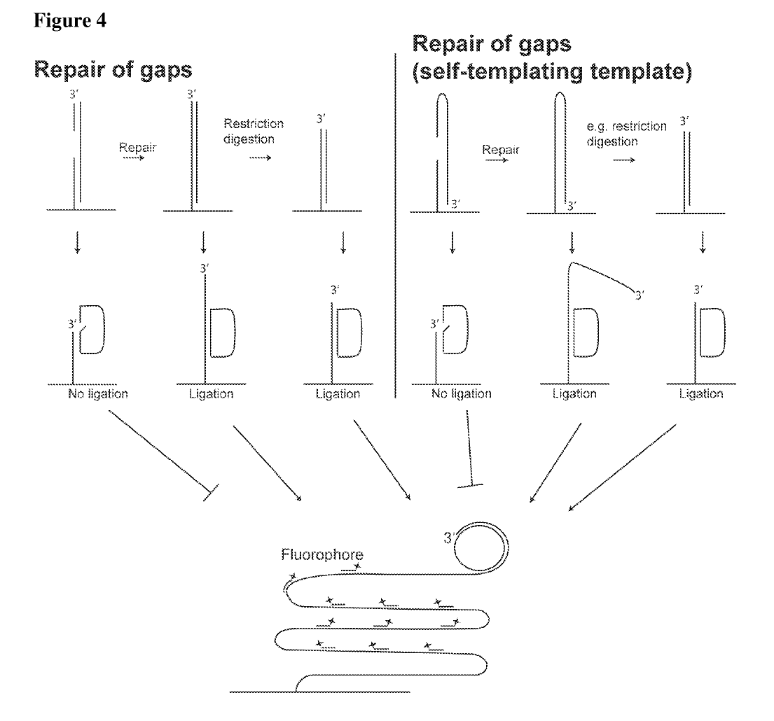

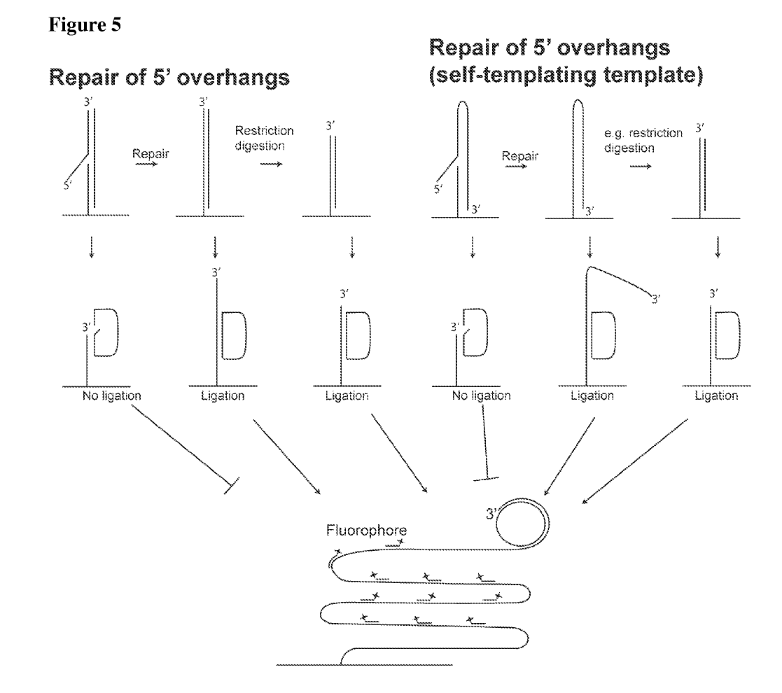

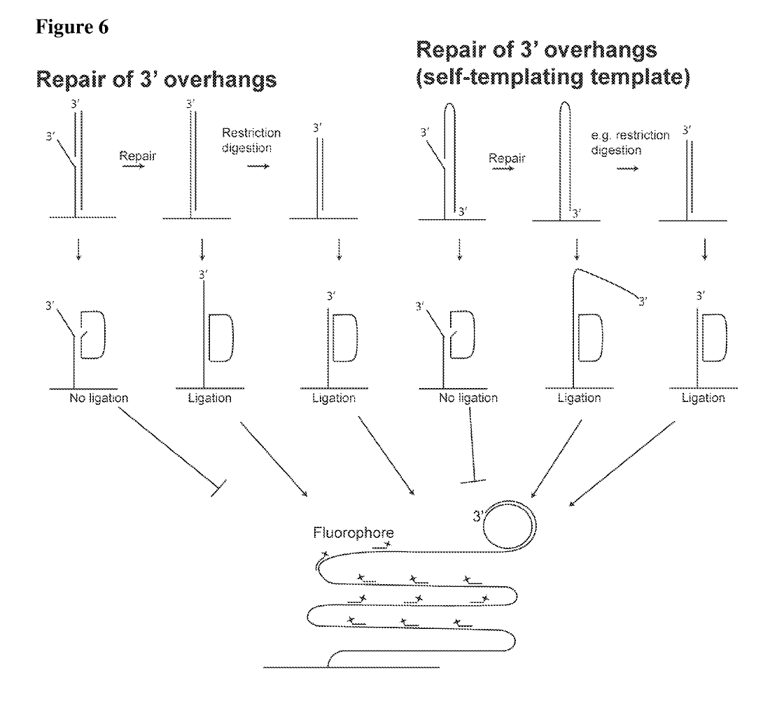

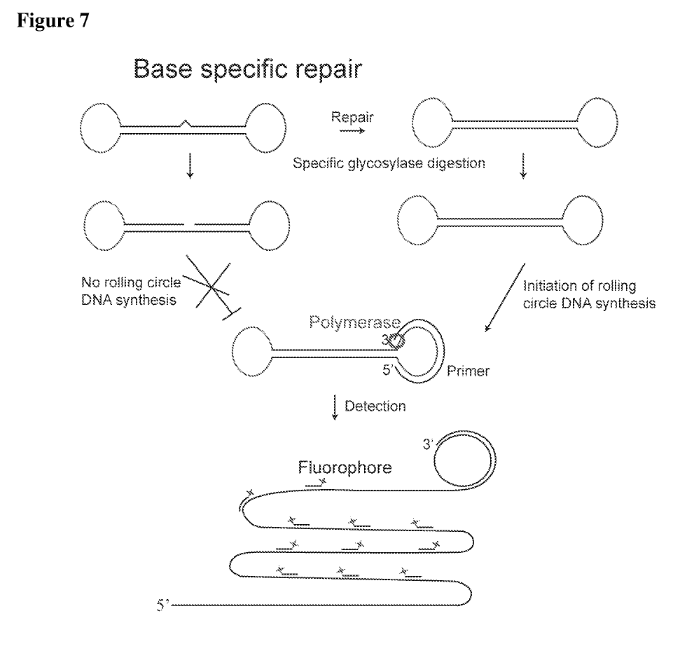

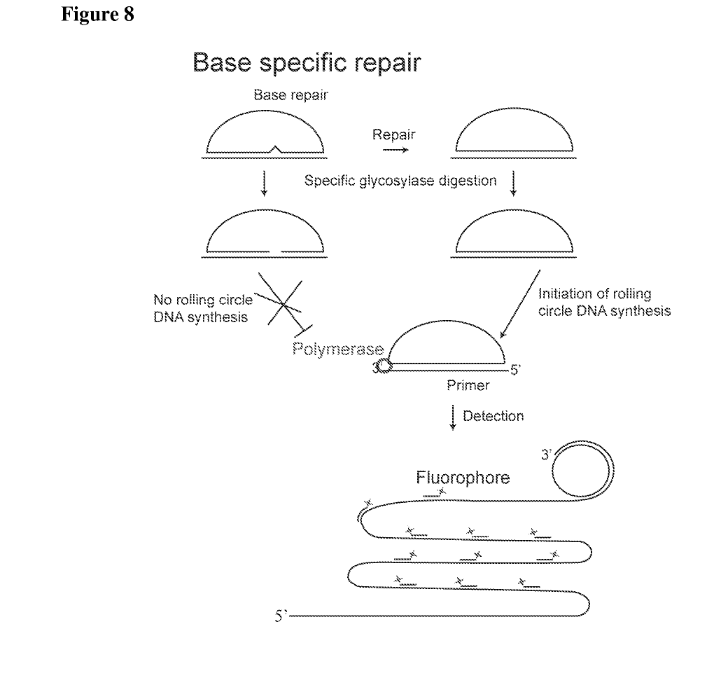

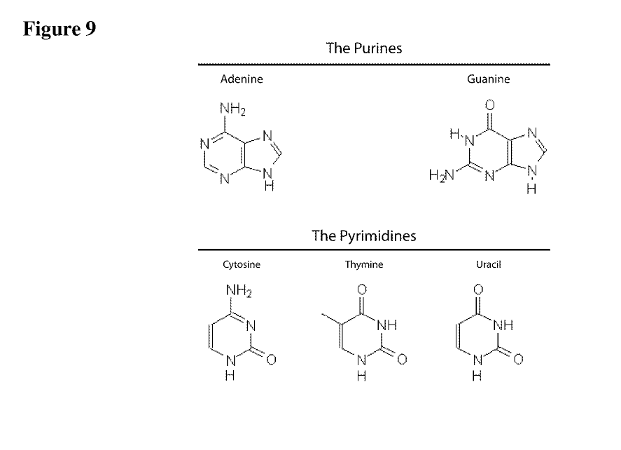

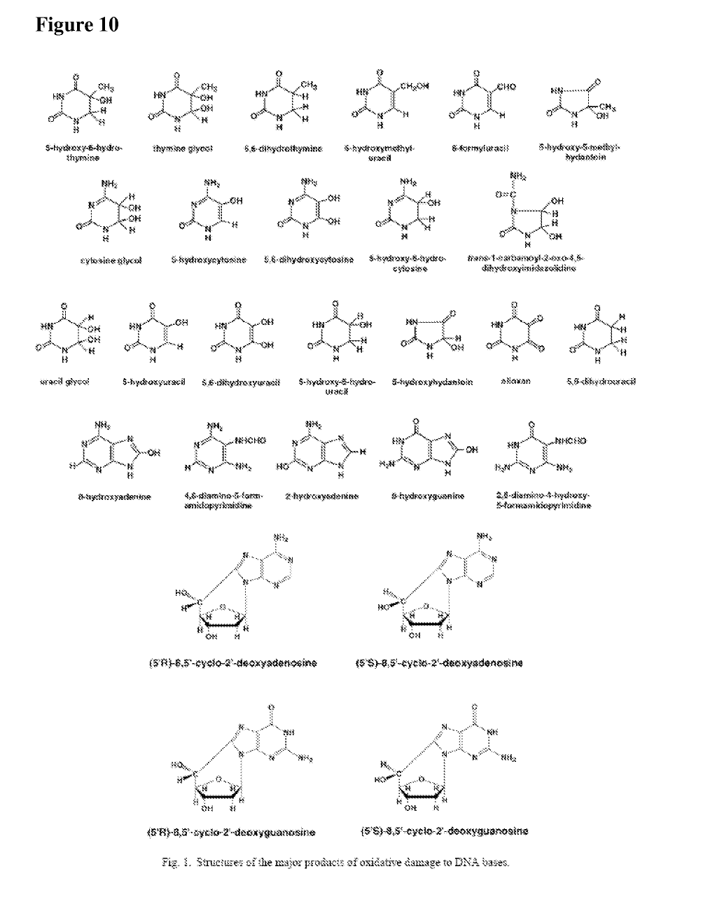

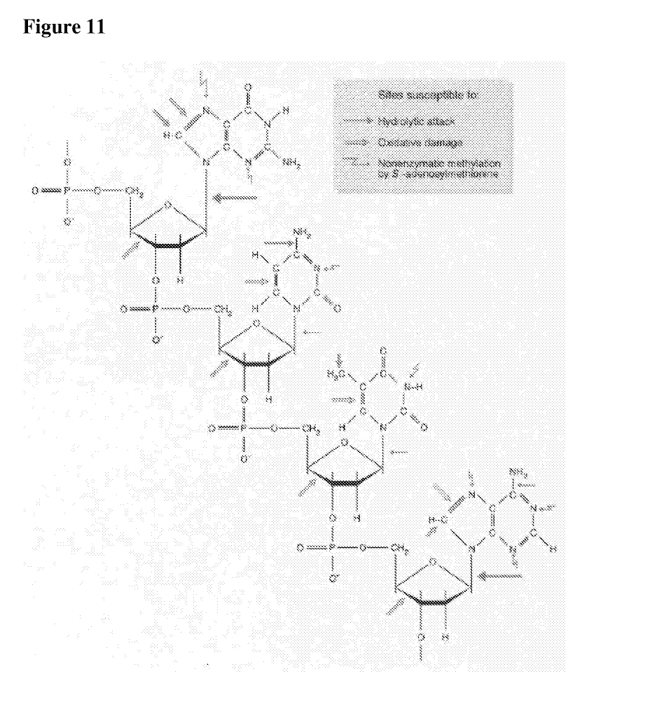

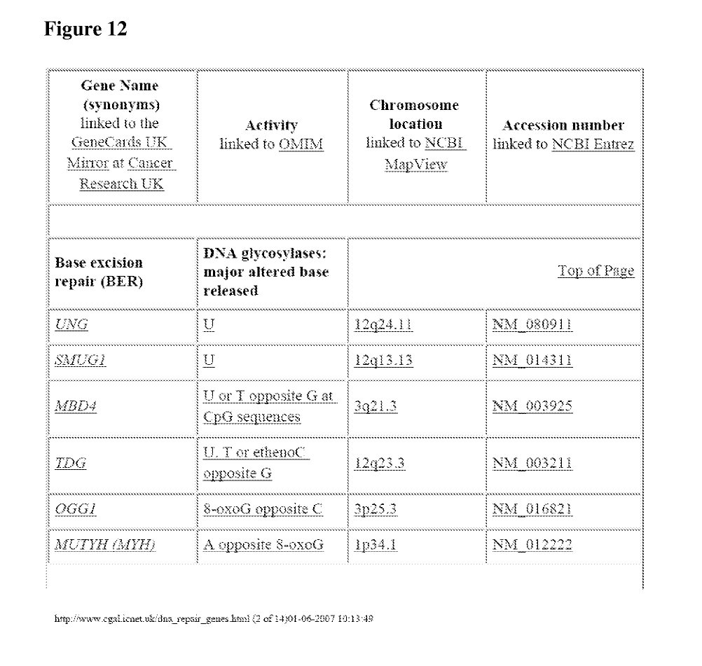

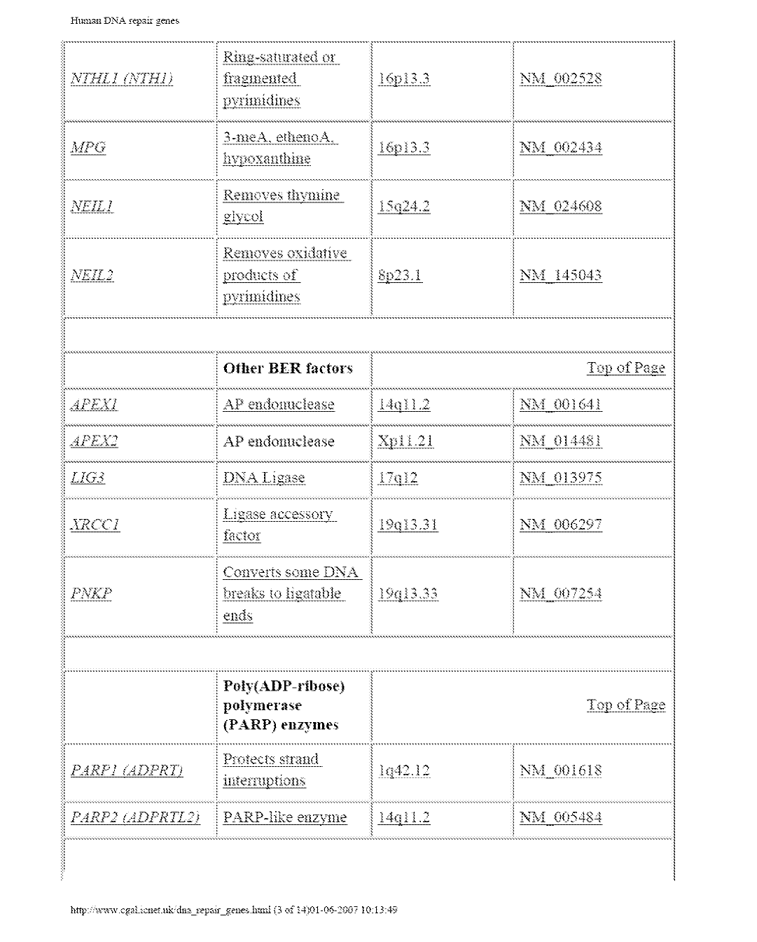

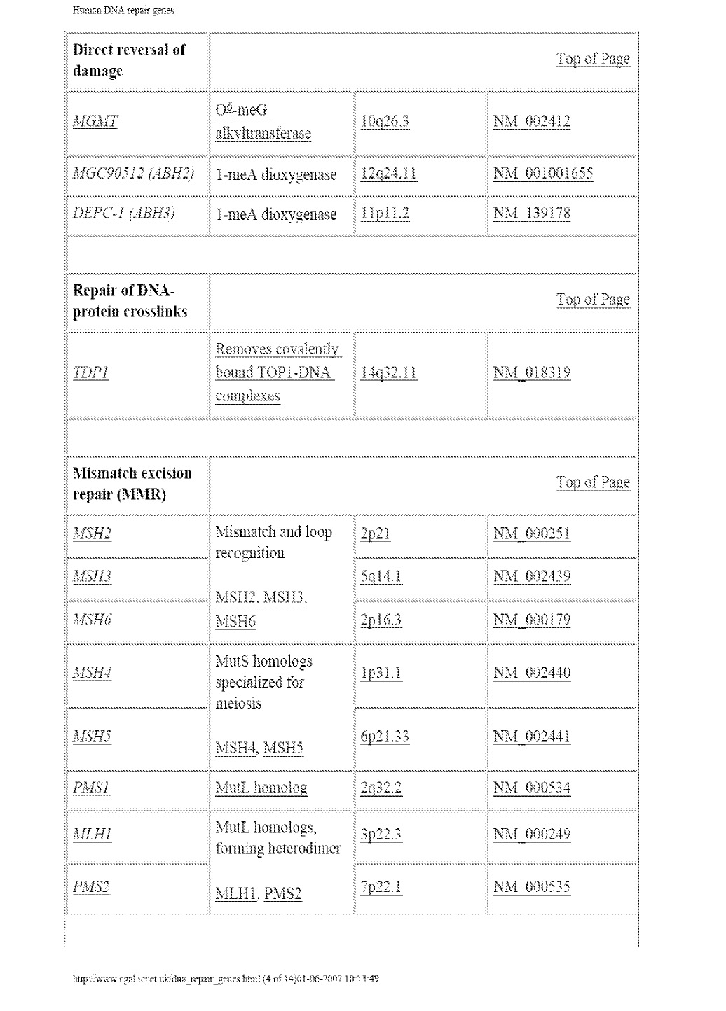

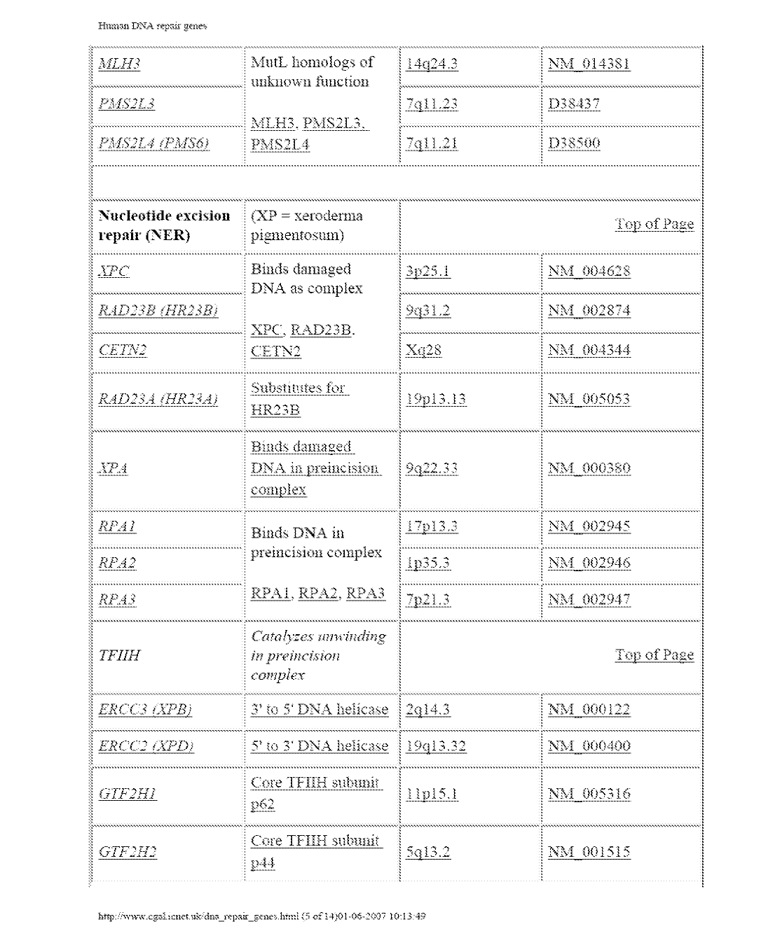

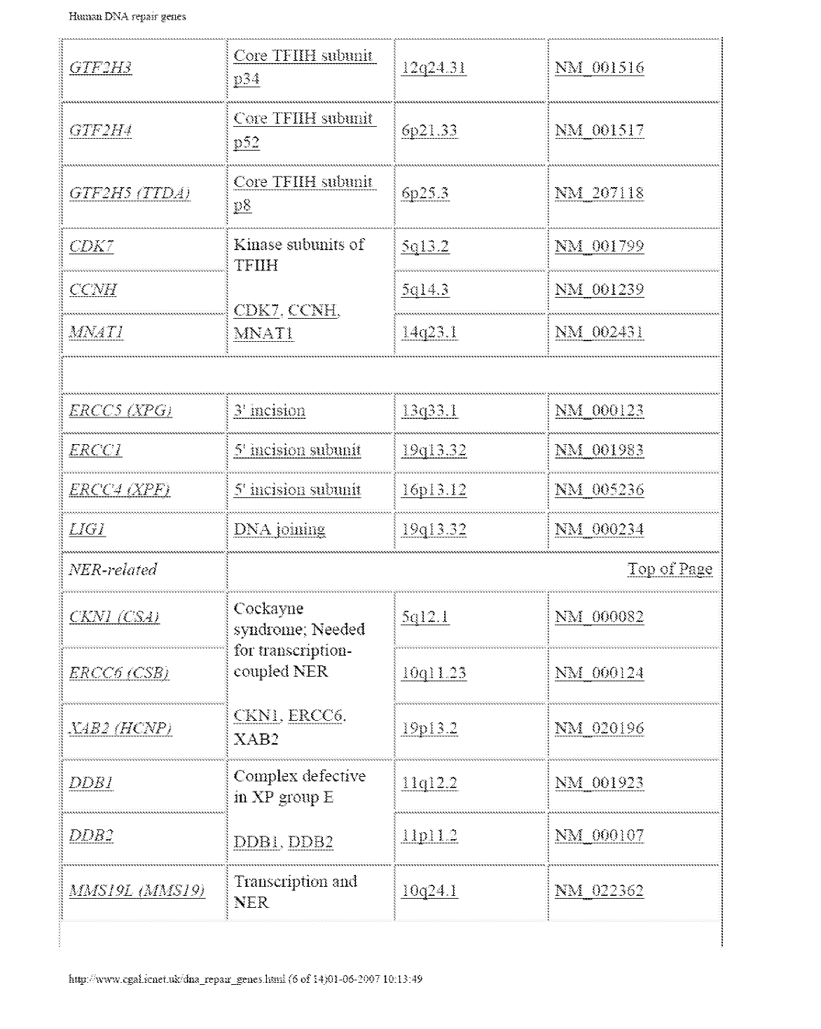

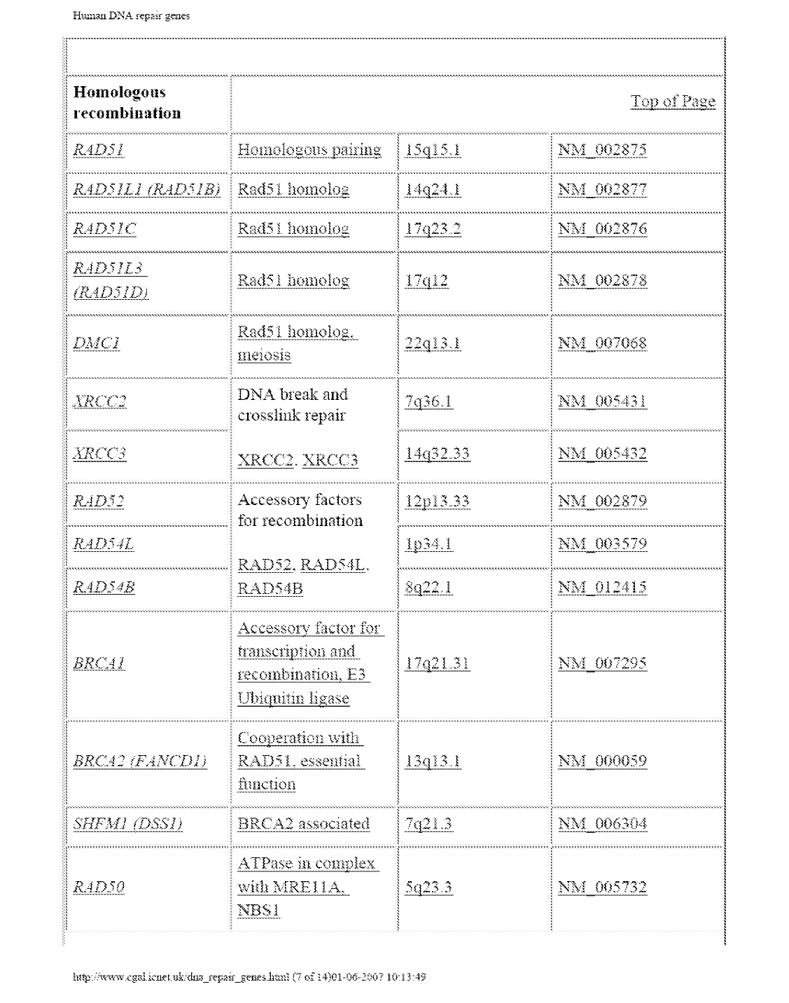

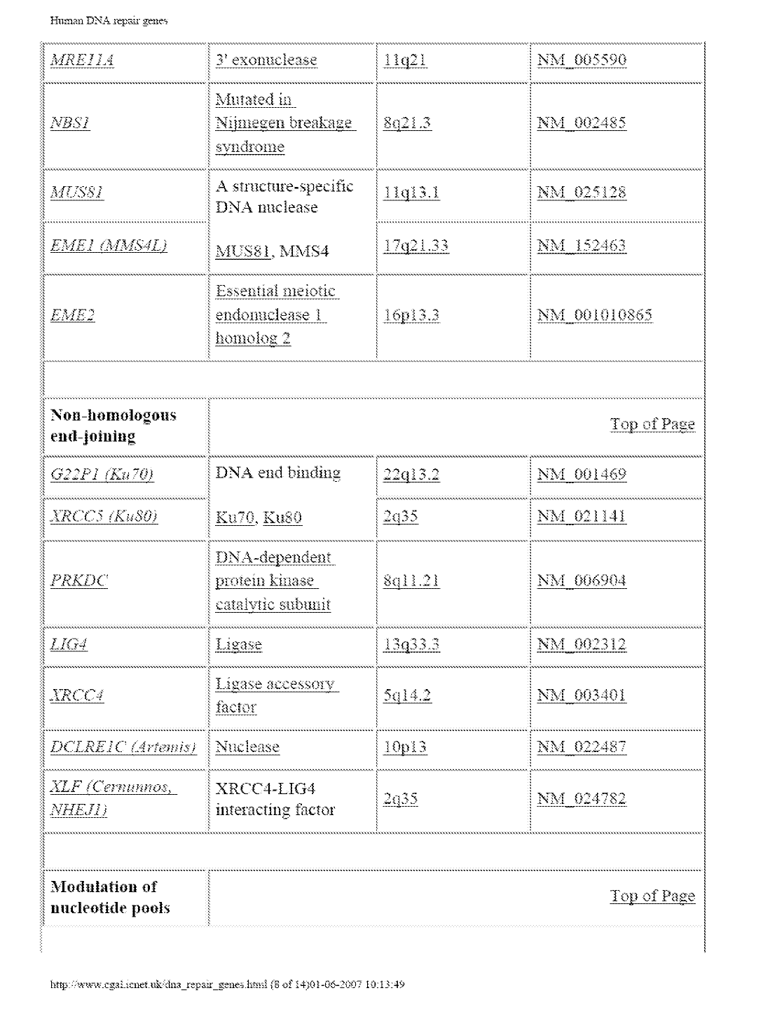

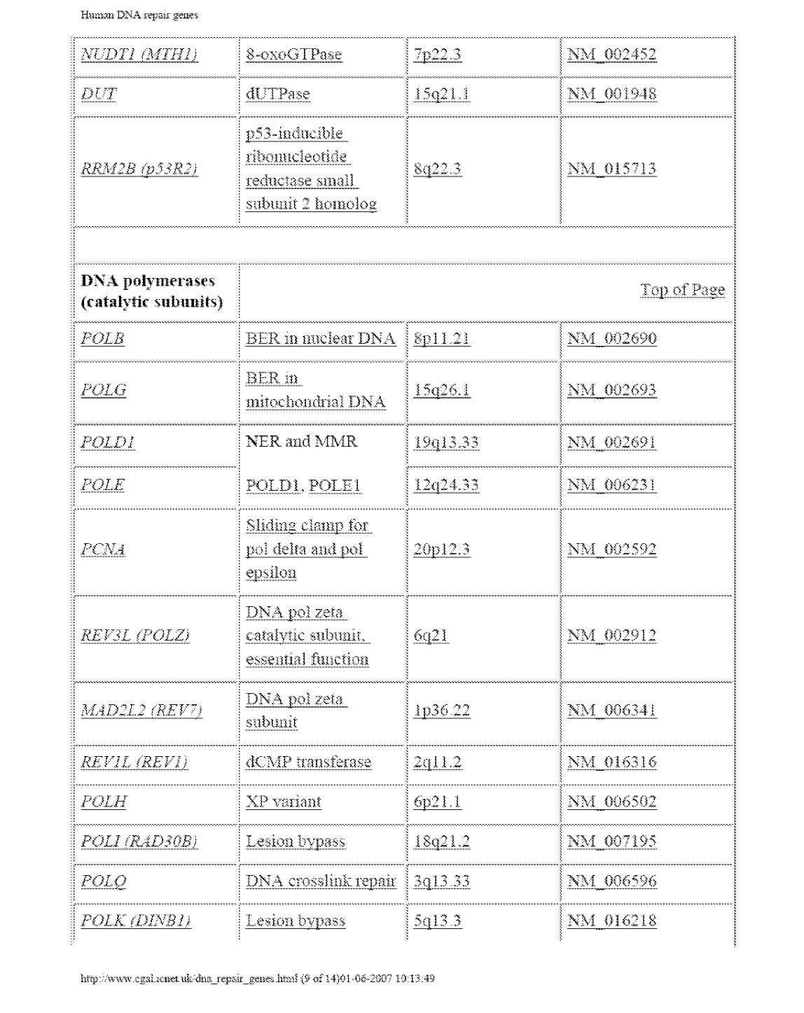

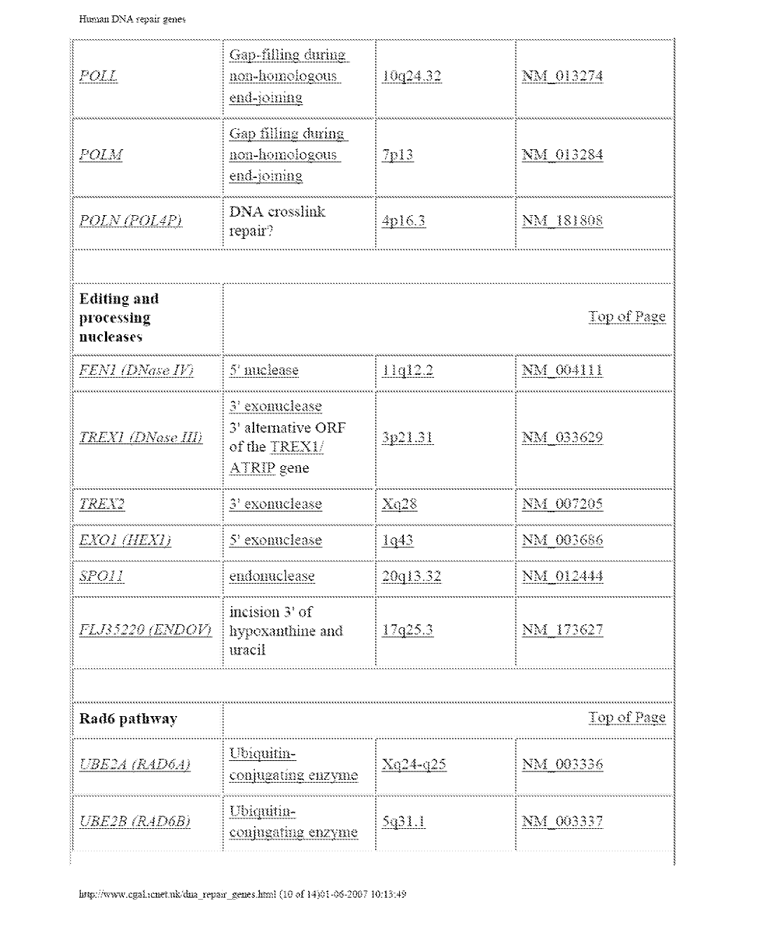

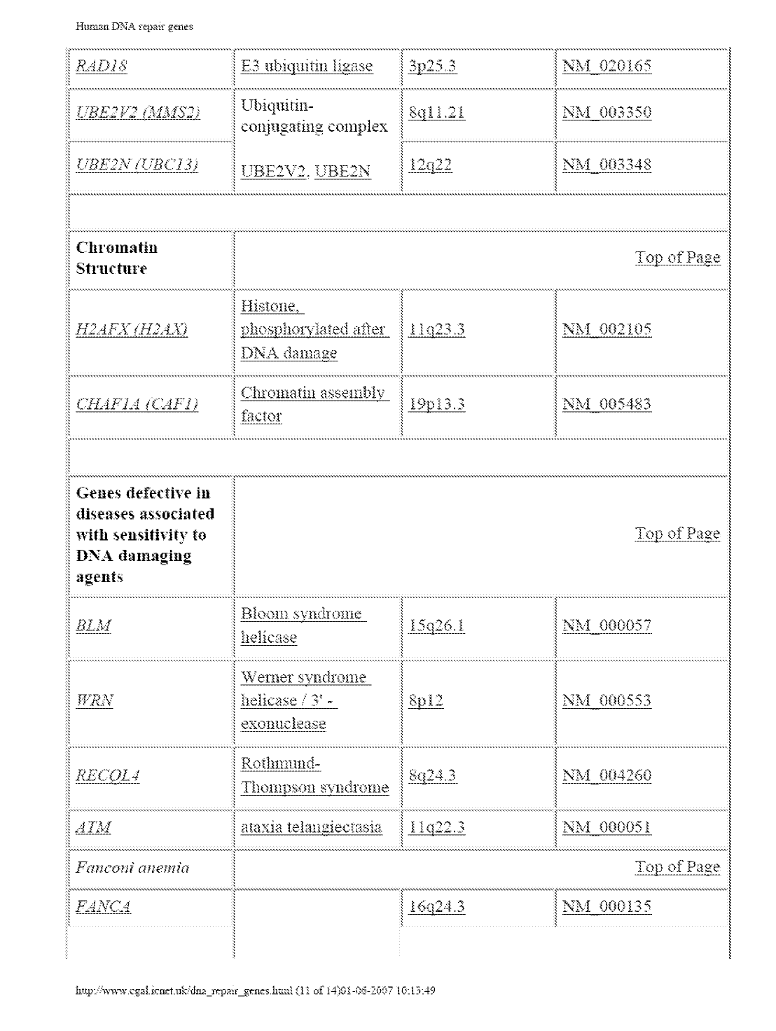

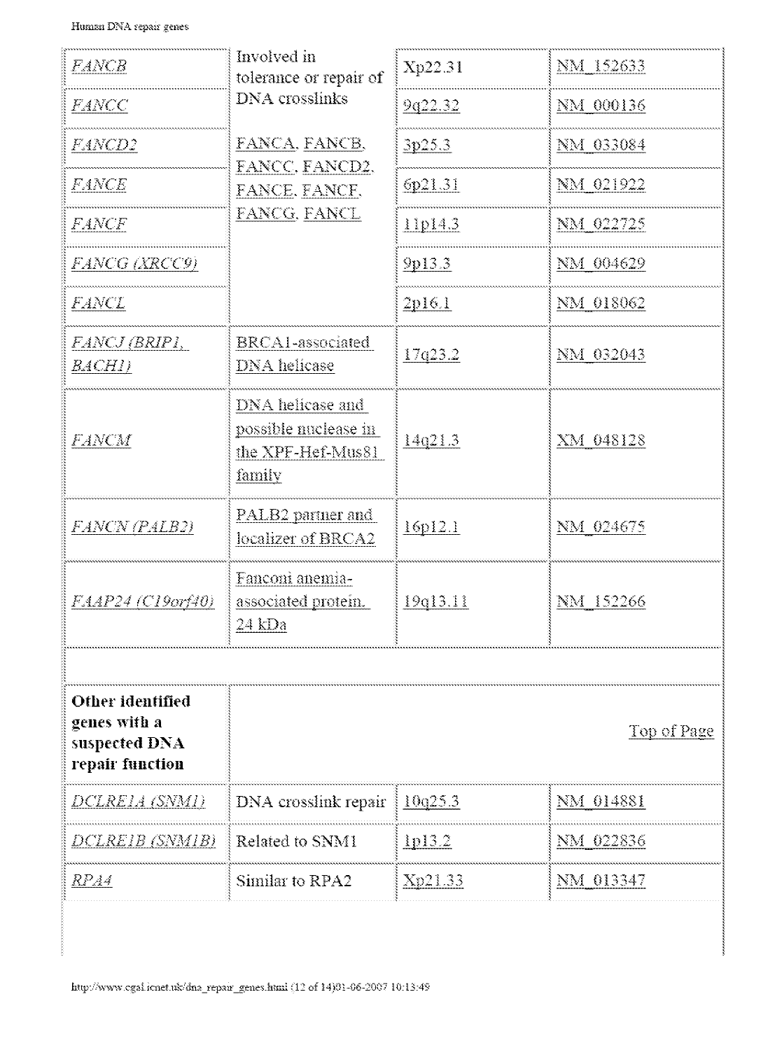

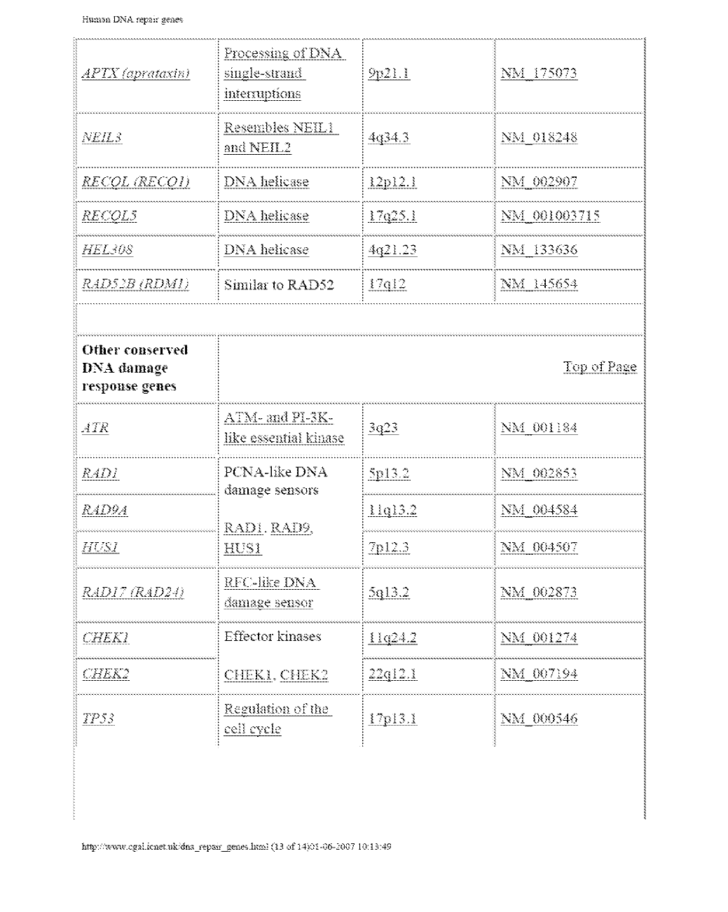

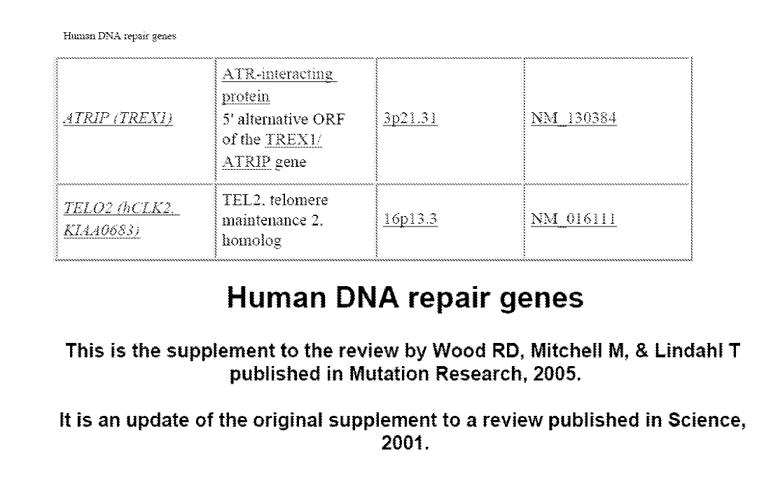

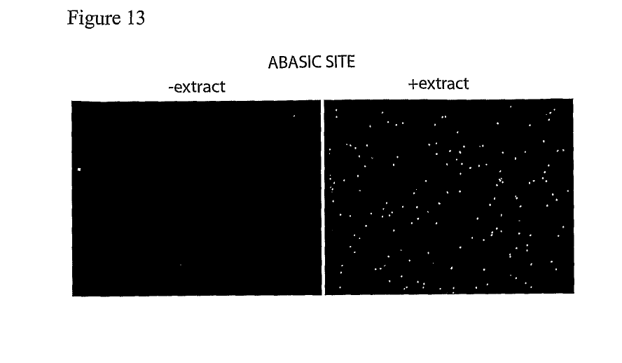

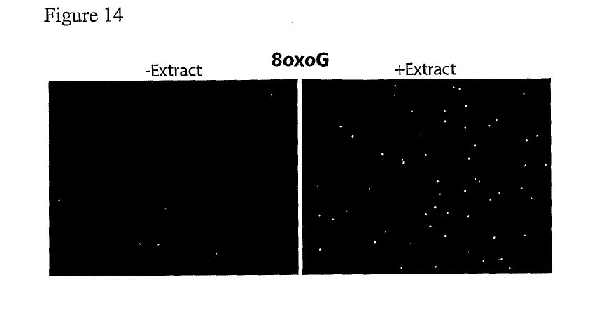

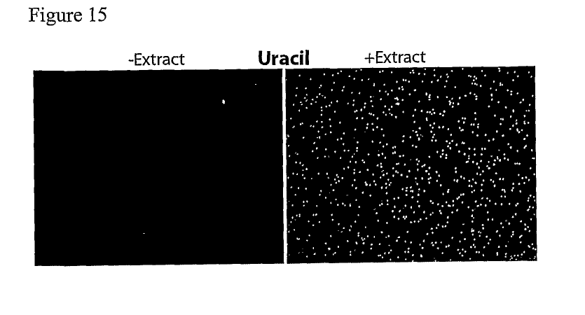

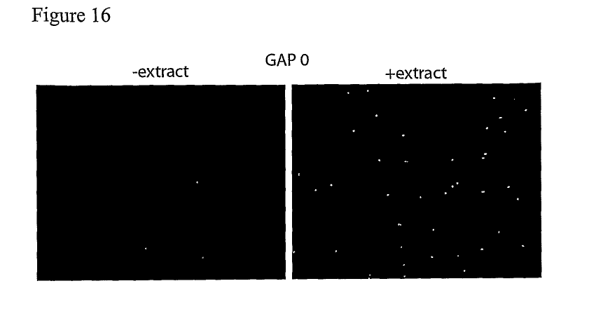

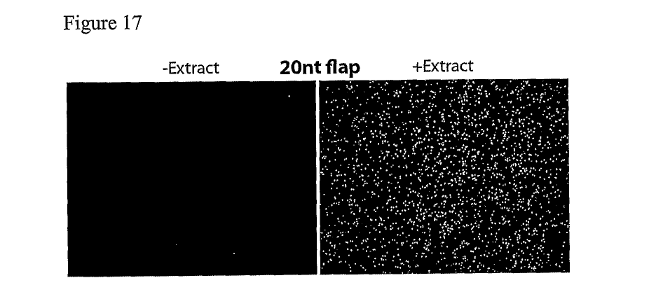

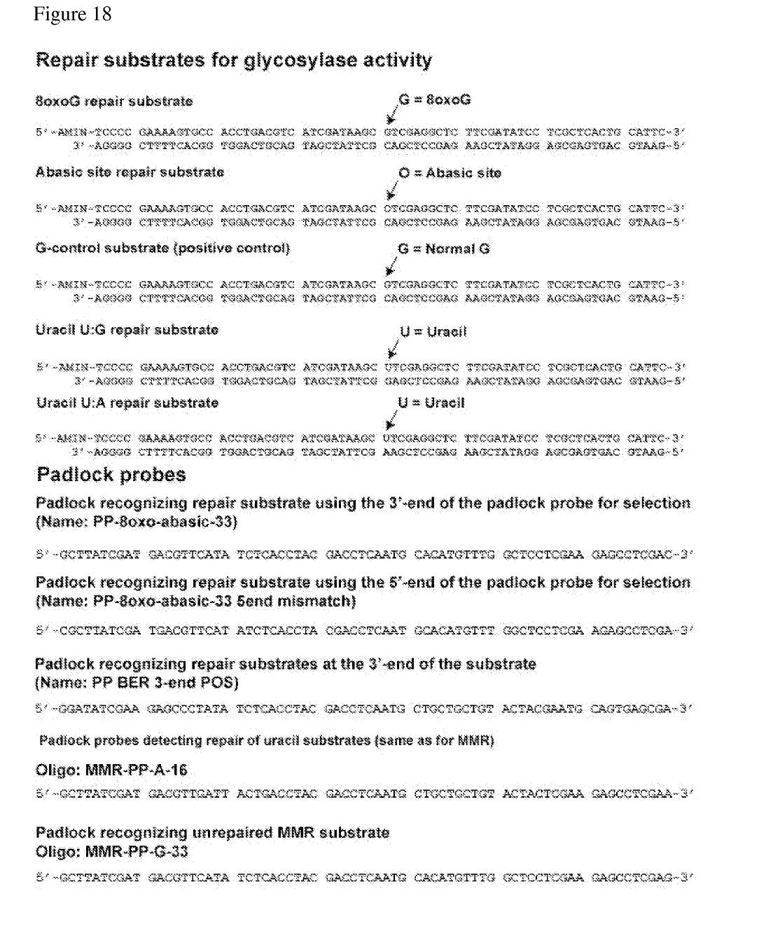

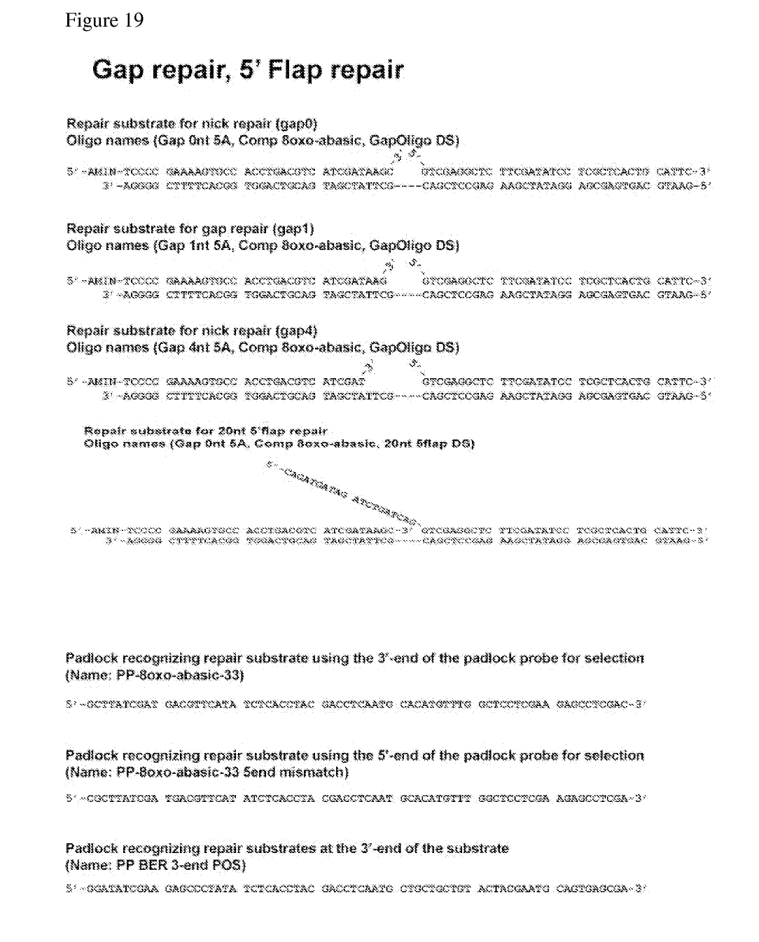

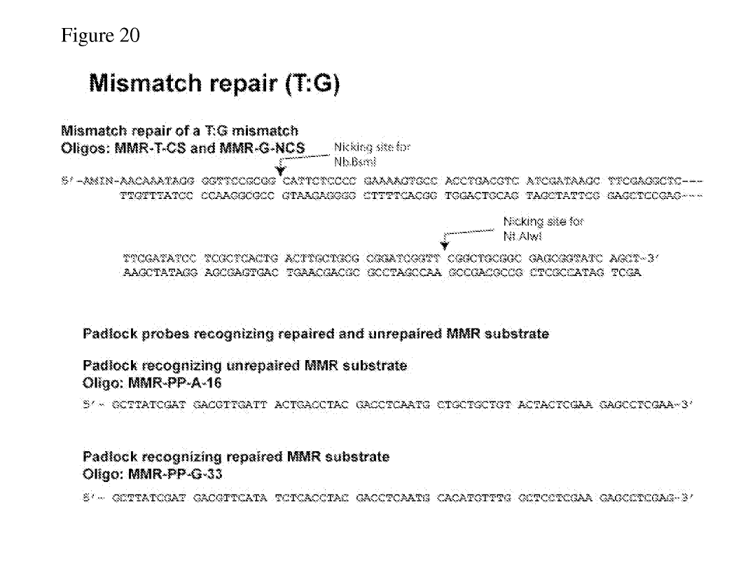

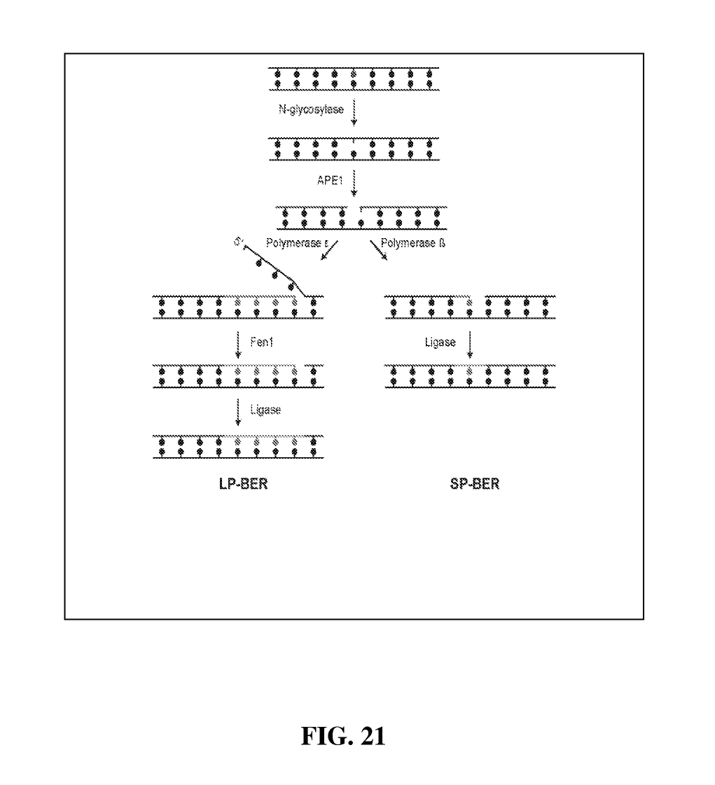

Detection of the repair of a double stranded substrate containing one abasic site opposite a cytosine by the method described in FIG. 1 left. Optionally, the abasic site can be cleaved by e.g. APE1 following incubation with cell extract, thereby minimizing background amplification. Sequences are provided in FIG. 18.

FIG. 14: Detection of the Repair of an 8oxoG in a Double Stranded Substrate.

Detection of the repair of a double stranded substrate containing one 8oxoG base paired to C by the method described in FIG. 1 left. Optionally, the abasic site can be cleaved by e.g. FPG following incubation with cell extract, thereby minimizing background amplification. Sequences are provided in FIG. 18.

FIG. 15: Detection of the Repair of a Uracil in a Double Stranded Substrate.

Detection of the repair of a double stranded substrate containing one uracil base paired to G by the method described in FIG. 1 left. Optionally, the abasic site can be cleaved by a combination of UNG and APE1 following incubation with cell extract, thereby minimizing background amplification. Sequences are provided in FIG. 18.

FIG. 16: Detection of the Repair of a Nick in a Double Stranded Substrate.

Detection of the repair of a double stranded substrate containing a nick by the method described in FIG. 4 left. Sequences are provided in FIG. 19.

FIG. 17: Detection of the Repair of a 20 Nucleotide 5′ Flap Overhang in a Double Stranded Substrate.

Detection of the repair of a double stranded substrate containing a 20 nucleotide 5′flap by the method described in FIG. 5 left. Sequences are provided in FIG. 19.

FIG. 18: Sequence List of Substrates and Padlock Probes used for the Detection of the Repair of Damages Nucleotides.

FIG. 19: Sequence List of Substrates and Padlock Probes used for the Detection of the Repair of Nicks (gap O) and Gaps of One and Four Nucleotides. Furthermore, sequences for the 20nt 5′ flap substrate are provided.

FIG. 20: Sequence List of Substrates and Padlock Probes used for the Detection of the repair of a T:G Mismatch.

### DETAILED DISCLOSURE OF THE INVENTION

Cells are constantly exposed to DNA damage caused by exposure to either endogenous reactive metabolites or exogenous damaging agents which can e.g. oxidize, deaminate, crosslink or alkylate DNA [4, 5]. One of the major products resulting from oxidation is 8-oxo-2′-deoxyguanosine (8-oxo-G), which is estimated to be generated 10⁴ to 10⁵ times per cell per day [6]. A list of oxidized bases is given in FIG. 10. What makes 8-oxo-G dangerous, if the base is not repaired, is that it can base pair with A, which can lead to G:C→A:T conversions following replication. Other major base damages are e.g. C→U transitions (a deamination), abasic sites (DNA apurinic/apyrimidinic (AP) sites occur as a consequence of nonenzymatic hydrolysis of base-sugar bonds in DNA and are also generated by DNA glycosylases as reaction intermediates in the BER pathway, in particular due to removal of uracil from misincorporated dUMP residues in DNA), methylations and formamidopyriminer [4]. Examples of damages caused by alkylation are O6-methylguanine (O6meG) and 3-methyladenine (3meA), 1-methyladenine (1meA), 3-methylcytosine (3meC), 1-ethyladenine and 1-6-ethenoadenine [5, 7].

Crosslinks are mainly generated by UV-B or UV-C light. Cyclobutane dimers between adjacent thymidines and/or cytosines comprise the majority of photolesions after UV-exposure with pyrimidine [6,4]-pyrimidone photoproducts generated in lesser amount. These covalent lesions distort the double helix and are clearly mutagenic, causing characteristic transitions known as “UV signature mutations”. Photolesions are repaired mainly by the nucleotide excision repair (NER) pathway [8]. Another group of enzymes which can repair UV-induced DNA damage is photolyases. Photolyases comprise efficient enzymes to remove the major UV-induced DNA lesions, cyclobutane pyrimidine dimers (CPDs) and 6-4 photoproducts (6-4PPs). While photolyases are present in all three kingdoms of life (i.e., bacteria, prokaryotes and eukaryotes), placental mammals appear to have lost these enzymes when they diverted from marsupials during evolution. Consequently, man and mice have to rely solely on the more complex and, for these lesions, less efficient nucleotide excision repair (NER) system [9].

The main pathway for correcting damaged bases is the DNA base excision repair (BER) pathway. Damaged bases are recognized by a DNA glycosylase which catalyzes the hydrolysis of the glycosidic bond between the modified base and the sugar moiety to release the base and generate an abasic site (apurinic/apyrimidic (AP) site). Numerous DNA glycosylases exist with different or overlapping substrate specificities [10, 11]. Subsequent to base removal, the AP endonuclease 1 (APE1) cleaves the backbone at the 5′-end of the AP site. From this point in the reaction two different mechanism are believed to be able to reconstitute the DNA strand, either the short patch BER (SP-BER) or the long patch BER (LP-BER) [12].

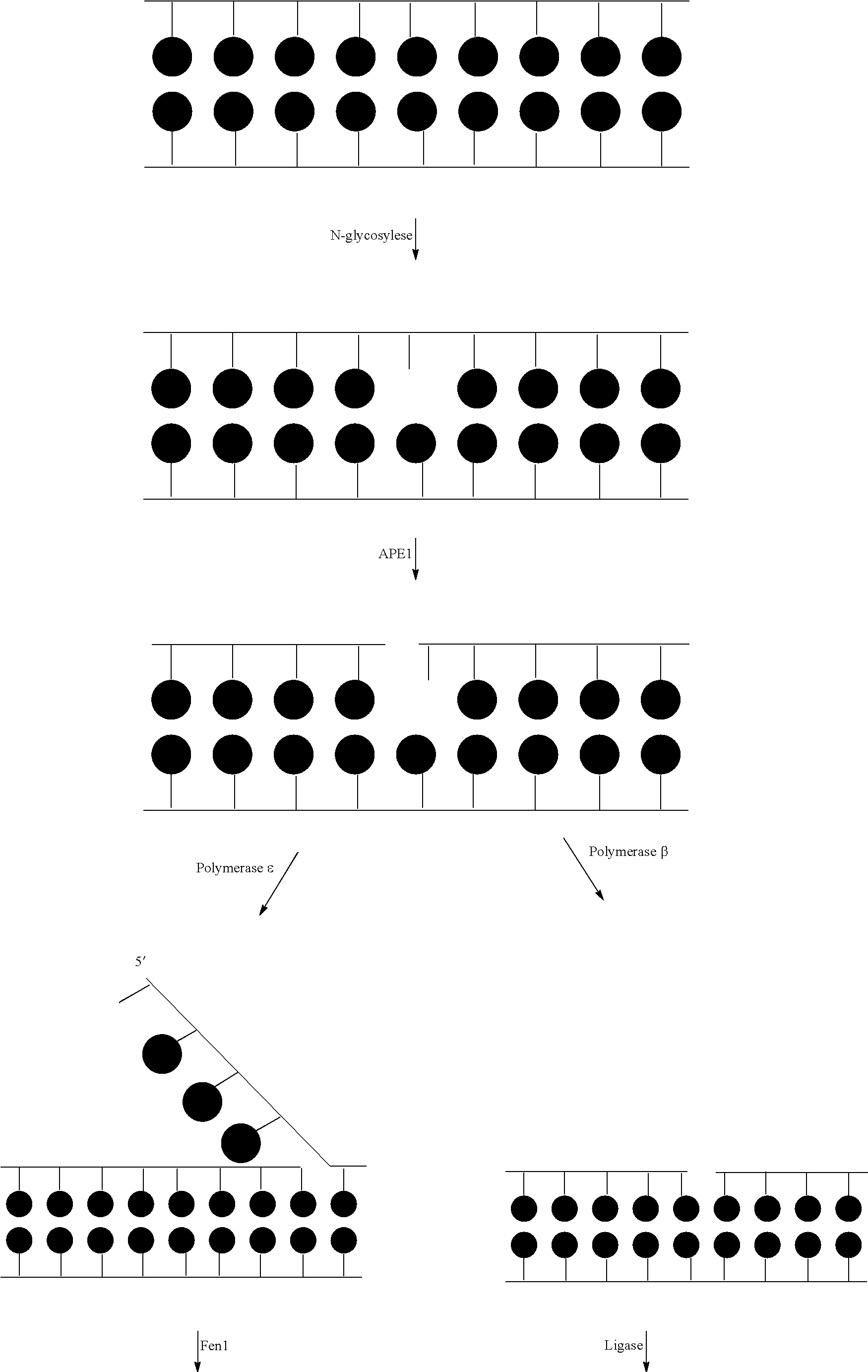

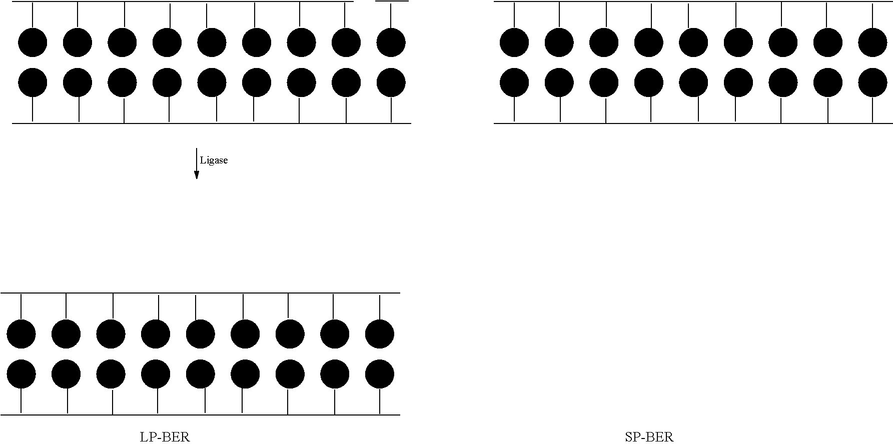

The above table illustrates LP-BER and SP-BER. Both pathways start with the removal of a damaged base by a glycosylase and APE1 makes a nick 5′ to the abasic site. In SP-BER DNA polymerase β removes the sugar group and inserts the missing nucleotide thereby preparing the substrate for ligation. In LP-BER polymerase ε makes strand displacement thereby inserting several nucleotides. Fen1 removes the displaced strand preparing the substrate for ligation.

In SP-BER, DNA polymerase β removes the abasic sugar residue and fills the gap, thereby preparing the DNA for ligation. However if the abasic sugar residue is reduced or oxidized it cannot be removed and is therefore subjected to LP-BER. In this case a polymerase extends from the nick and displaces the downstream nucleotides thereby creating a 5′-flap which can be removed by Fen1 followed by ligation [10].

Several proteins are known to be involved in the repair of different DNA lesions such as base excision repair (BER), direct reversal of damages, repair if protein-DNA crosslinks, mismatch excision repair (MMR), nucleotide excision repair (NER), homologous recombination, and non-homologous end-joining. Furthermore, repair often also involves polymerases, editing and processing nucleases and numerous other proteins known to be involved in DNA repair. A list of proteins (gene names) known to be involved in one or more repair events together with their activity, chromosome location and NCBI accession number are listed in FIG. 12.

Detection of Repair Events on DNA Substrates using Padlock Probes

By having a double stranded substrate containing an unprocessed substrate moiety, the repair of the substrate moiety can be monitored. This can be done by incubating said substrate with a sample containing the one or more enzymes necessary to repair the unprocessed substrate moiety. Following incubation with said sample the double stranded substrate can be made single stranded by denaturation or exonuclease digestion, leaving only the strand which contained the unprocessed substrate moiety.

By hybridizing and ligating a padlock probe to the repaired/un-repaired substrate moiety the repair can be monitored. Only if the unprocessed substrate moiety has been correctly repaired will the padlock probe be ligated. Following ligation the padlock probe can be amplified by rolling circle DNA synthesis using e.g. the 3′-end of the coupled strand as primer.

Thus, in one aspect of the invention, the invention relates to a method for the detection of DNA repair using double stranded substrates containing one or more damages, wherein the repair of said damage can be monitored using padlock probes and rolling circle DNA synthesis.

In a preferred embodiment the one strand of the double stranded substrate is coupled to a solid support, allowing for stringent washing between individual steps and for separation of the two strands on the double stranded substrate. Furthermore, solid support rolling circle replication can be performed.

Thus in a preferred embodiment the invention relates to a double stranded substrate which is coupled to a support through binding of the 5′-end.

The Substrate:

The substrates of the invention are comprising one or more individual nucleic acid sequences. The substrate can comprise any sequence of the natural nucleotides G, C, A, T, I, U, or any artificial nucleotides e.g., but not limited to, iso-dCTP, iso-dGTP, nucleotides which are substrates for glycosylases or a mixture thereof.

Several modified nucleotides, corresponding to cellular DNA lesions, are commercially available [13]. The one or more individual nucleic acid sequences of the substrates of the invention have a linear length of 10-1000 nucleotides. Thus, in one aspect, the invention relates to a method, wherein the one or more substrates have a length of 20-500 nucleotides, such as e.g. 20-150 nucleotides, or such as e.g. 30-300 nucleotides, or such as e.g. 50-150 nucleotides, or such as e.g. 80-150 nucleotides, or such as e.g. 100-120 nucleotides. The substrates can be synthesized by standard chemical methods (e.g. beta-cyanoethyl phosphoramidite chemistry). If long oligonucleotides are needed they can e.g. be made by ligating two or more oligonucleotides. For coupling one or more of the oligonucleotides can contain an amin, biotin, phosphate group, or another group allowing coupling to a solid support.

The oligonucleotide which is going to prime the rolling circle DNA synthesis, in the method of the invention, can be anchored to a solid support, thereby attaching the following rolling circle product to a surface. This will make it easier to change buffer conditions, and improve washing between the different steps, thereby minimizing background. In one example, the primer can be coupled in the 5′-end to a solid support—a 5′-biotin labeled primer may e.g. be coupled to a streptavidine coated solid support including, but not limited to, PCR-tubes, ELISA plates, beads, plastic CDs (e.g. produced by the company Åmic), and microscope slides. In another example the primer is coupled to a solid support through a 5′-amin, thereby getting a covalent linkage, including, but not limited to, PCR-tubes, ELISA plates, beads, plastic CDs (produced by the company Åmic), and microscope slides. It is to be understood that the primer can also be coupled to a surface when it is part of the substrate. Thus, in one aspect, the invention relates to a method, wherein the primer is immobilized on a solid support. Alternatively, the substrate is coupled to a support following incubation with a sample. Furthermore, blocking of one or more ends of the substrate by one or more modifications could protect the probe from exonucleolytic digestion.

Blocking for 5′-Exonuclease Activity:

By positioning one or more modified nucleobases, backbone units, internucleoside linkers or sugar moieties at one or more of the 5′-ends, 5′-exonucleolytically degradation could be inhibited, whereas the 5′-endonuclease activity would likely not be blocked of enzymes such as DNA2P, exo1 and Fen1. Furthermore, by positioning a cap on the 5′-end inhibiting ligation, unspecific ligation could be inhibited minimizing false positive signals. This ligase activity could, besides be caused by ligase, also be caused by e.g. topoisomerase I.

Thus, in one embodiment, one or more of the 5′ ends of the double stranded oligonucleotide probe are capped with a modification selected from the group, but not limited to PO₃, CH₃, a C-linker, NH₃, CH₂CH ₃, biotin or H.

Thus, in a second embodiment, one or more of the 5′-ends comprises one or more modified nucleobases, backbone units, internucleoside linkers or sugar moieties inhibiting exonucleolytically degradation.

Blocking for 3′-Exonuclease Activity:

By positioning one or more modified nucleobases, backbone units, internucleoside linkers or sugar moieties at one or more of the 3′-ends, 3′-exonucleolytically degradation could be inhibited, whereas 3′-endonuclease activity would likely not be blocked, Furthermore, by positioting a cap on the 3′-end inhibiting ligation, unspecific ligation could inhibited. This ligase activity could, besides be caused by ligase, also be caused by e.g. a topoisomerase II.

Thus, in one embodiment, one or more of the 3′-ends are capped with a modification selected from the group, but not limited to, PO₃, CH₃, a C-linker, NH ₃, CH ₂CH₃, biotin or H.

Thus, in a second embodiment, one or more of the 3′-ends comprise one or more modified nucleobases, backbone units, internucleoside linkers or sugar moieties inhibiting exonucleolytic degradation.

The number of modifications should be, but not limited to, such as 1-10 modifications, such as 3-5 modifications, or such as 3 modifications.

Examples of Probe Designs and Method a Described in FIGS. 1-6

The substrate can be provided in several formats: In one format the substrate, which consists of a single oligonucleotide, through self-templated hybridization is able to form a region which can be a substrate moiety for one or more enzymes. The advantage of this setup is that the number of free ends are limited, thereby avoiding exonucleolytic degradation. In another format the substrate, which consists of more than one oligonucleotide, through hybridization, is able to form a region which can be a substrate moiety for one or more enzymes. The advantage of this system is that one or more nicks can be positioned in the substrate, which is known to induce some repair events (see background of the invention). It is to be understood that all of the following sections refers to both formats of the substrate.

Substrate Moiety

The moiety which is going to be repaired is selected from the group, but not limited to: Mismatches, loop (2-100 nucleotides), damaged bases, damaged sugar groups, damaged internucleoside linkers, internucleotide crosslinks, abasic sites, methylated bases, nicks, gaps 5′-overhangs, 3′-overhangs and chemical groups mimicking of the previous members of this group.

Substrate Incubation with Sample

A sample can be provided in several formats such as, but not limited to, cells grown on a surface, cells in solution, cell extracts, tissue preparations or purified enzymes. If the sample is cells on a surface or tissue sections, the substrate mixture can be provided by placing the substrate mixture in a liquid phase on the cells or tissue sections. Following substrate incubation the mixture can be transferred to a solid support before amplification or the amplification can be performed directly on the cell or tissue. If a penetration step is necessary to get the enzymes out of the cells or substrates into the cells a penetration step may be an advantage. This can e.g. be done by hypotonic treatment, detergents, electrophoration, or proteases.

Detergents, such as NP40, triton x-100, tween 20 may be used in the present invention. When using NP40, triton x-100 or tween 20, the concentration is preferably in the range of from about 0.01 to about 2%, from about 0.01 to about 0.05%, from about 0.05 to about 0.1%, from about 0.1 to about 0.2%, from about 0.2 to about 0.3%, from about 0.3 to about 0.4%, from about 0.4 to about 0.5%, from about 0.5 to about 0.6%, from about 0.6 to about 0.7%, from about 0.7 to about 0.8%, from about 0.8 to about 0.9%, from about 0.9 to about 1%, from about 1 to about 1.1%, from about 1.1 to about 1.2%, from about 1.2 to about 1.3%, from about 1.3 to about 1.4%, from about 1.4 to about 1.5%, from about 1.5 to about 1.6%, from about 1.6 to about 1.7%, from about 1.7 to about 1.8%, from about 1.8 to about 1.9%, from about 1.9 to about 2%. SDS can also be used as detergent, albeit at a lower concentration, such as from about 0.001 to about 1%

Cells can also be opened by repeated cycles of freezing and thawing. In a preferred embodiment two cycles of −80° C. to room temperature are performed

Optionally one or more enzymes or chemicals can be added to the sample to stimulate or inhibit the repair event. This could be enzymes such as kinases, ligases, polymerases glycosylases, nucleases, accessory proteins and cofactors or chemicals such as stimulators, inhibitors, NAD+, ATP and dNTPs.

Providing a 3′-End in Proximity of the Padlock Probe

To bring the 3′-end of the primer closer to the sequence part where the padlock probe is going to be hybridized, it could be an advantage to cleave the double stranded product with a restriction enzyme close to the padlock probe hybridization region. This step should preferably be performed following sample incubation and before padlock probe hybridization. This optional additional step will likely favor the rolling circle DNA synthesis step, because the 3′-end of the coupled oligonucleotide is going to be used as a primer for the amplification reaction. Therefore the applied polymerase for the rolling circle DNA synthesis reaction only needs limited (if any) 3′ exonuclease activity to start the amplification reaction.

Furthermore, if the original 3′ end is blocked to prevent exonucleolytic degradation of the probe, this blocking may also inhibit priming from this 3′-end, and thus another 3′-end is needed to initiate the rolling circle replication of the ligated padlock probe Thus in one embodiment of the invention the double stranded oligonucleotide probe comprises one or more recognition sites for one or more restriction enzymes.

Making the Double Stranded Substrate Single Stranded

Following sample incubation it is preferably to have a single stranded substrate for the padlock probe, therefore it is an advantage to remove one strand of the substrate. This can be done in numerous ways. If the substrate is coupled to a surface the substrate can be denatured by heating e.g. 5 min at 95° C., or by using 0.1M NaOH for a suitable time, such as from 1 to 60 min, for example 5 to 15 min at room temperature. NaOH is known to denature double stranded DNA. Alternatively, a 5′-exonuclease can be used e.g. lambda exonuclease. If one of the strands in the substrate is coupled to a surface through a 5′-end coupling only the uncoupled strand will be digested.

Padlock Probe Hybridization and Ligation

Padlock probe: A class of probes, comprising a nucleic acid sequence with a free 3′-end and a free 5′-end, which upon hybridization to its target will fold so that the 3′-end and the 5′-end are positioned next to each other, enabling ligation to form a closed circular structure. The size of the padlock probe may vary and is often determined by the practicalities associated with a particular assay. The skilled person will know how to evaluate the size of a specific padlock probe needed for a particular assay. Preferred padlock probes are preferably smaller than 1000 nucleotides and they are preferably between 30-150 nucleotides, such as e.g. 30-120 nucleotides, such as e.g. 30-100 nucleotides, such as e.g. 30-80 nucleotides, such as e.g. 50-80 nucleotides. The two hybridizing arms of the padlock probe are preferably between 5-30 nucleotides each, such as e.g. 5-25 nucleotides, such as e.g. 5-20 nucleotides, such as e.g. 5-15 nucleotides, such as e.g. 5-10 nucleotides. The padlock probe can comprise any sequence of the natural nucleotides G, C, A, T, I, U, or any artificial nucleotides e.g., but not limited to, iso-dCTP, iso-dGTP, or a mixture thereof. The padlock probes can be synthesized by standard chemical methods (e.g. beta-cyanoethyl phosphoramidite chemistry).

For the distinction of damaged nucleotides such as abasic sites, 8oxoG and uracil, the nucleotide at the 3′end of the padlock is preferably positioned opposite the damaged nucleotide. Alternatively, the 5′end is used. To increase the selection between a repaired and an un-repaired nucleotide the padlock probe can be further destabilized by introducing point mutations in the hybridizing arms of the padlock probe. Destabilization can also be introduced by e.g. incorporating artificial nucleotides in the hybridizing arms e.g. such as inosine or abasic sites.

A padlock probe can be hybridized and ligated in one step or the two steps can be separated. The two steps can be performed simultaneously or sequentially in a buffer containing 1× ligase buffer (Fermentas), less than 2000 mM NaCl, such as 250 mM NaCl or 500 mM NaCl, 0.0001-1 mM ATP and 0.0001-0.5 unit T4 DNA ligase (Fermentas) for 30 min at 37° C. Alternatively, a thermostable ligase (e.g. Ampligase) is used together with an appropriate energy-source allowing for denaturation, padlock probe hybridization and ligation in one step. Both T4 DNA ligase and Ampligase have a very high discrepancy for correct base pairing at the point of ligation.

If hybridization and ligation are separated in two independent steps, the hybridization can be performed in a standard buffer containing from 0-2M NaCl, 0-50% formamide and with or without carrier DNA or RNA. In the ligation step preferably T4 DNA ligase is used in conditions containing from 0-1M NaCl, such as 100-800 mM, for example 250-500 mM NaCl.

Thus, if the damaged nucleotide position in the substrate has not been repaired the padlock probe cannot be circularized.

Preferably the padlock is hybridized close to a 3′-end enabling efficient rolling circle DNA synthesis. Thus, in one embodiment, the invention relates to a method, wherein said circular nucleic acid probe hybridizes 100 nucleotides or less from the 3′-end of the target nucleic acid molecule, such as e.g. 0-100 nucleotides, such as e.g. 0-75 nucleotides, such as e.g. 0-50 nucleotides, such as e.g. 0-25 nucleotides, or such as e.g. 0-20 nucleotides, or such as e.g. 0-15 nucleotides, or such as e.g. 0-10 nucleotides, or such as e.g. 0-5 nucleotides, or such as e.g. 4 nucleotides, or such as e.g. 3 nucleotides, or such as e.g. 2 nucleotides, or such as e.g. 1 nucleotide, or such as e.g. 0 nucleotides.

Rolling Circle DNA Synthesis

When a polymerase and deoxynucleoside triphosphates (dNTPs) are combined with a probe hybridized to a primer (primer 1) under correct buffer conditions, rolling circle replication can take place. The polymerase will start the polymerization from the 3′-end of the primer, using the circular probe as a rolling-circle-template. As the circular probe is endless, the rolling circle product will comprise a multimer complementary to the sequence of the circular probe. Preferably the polymerase is the Phi29 DNA polymerase. A final concentration of 0.001-2 units of phi29 polymerase (Fermentas) is used, preferably 0.05-1 unit is used. A final dNTP concentration of 0.005-10 mM, preferably 0.1-1 mM is used. Alternatively, other polymerases such as, but not limited to, the T7 DNA polymerase, Sequenase Version 2.0 T7 DNA Polymerase, and Bst DNA polymerase can be used. The incubation time should be between 10 minutes and 24 hours, preferably 30 minutes to 5 hours, at the temperature optimal for the polymerase of choice. For some of the polymerases addition of single stranded binding protein (SSB) enhances the rolling circle activity. Since the Phi29 DNA polymerase is not enhanced by SSB, a concentration of 0 μg/μl SSB is preferably used. Alternatively a concentration of 0.001-0.2 μg/μl can be used. The length of the rolling circle product is preferably between 500 and 500.000 nucleotides in length. The speed and duration of the elongation can be controlled by varying the concentrations of dNTP, polymerase, circle, primer, and SSB. Furthermore, temperature and buffer conditions are adjustable. Following rolling circle from a solid support, it can be washed as described above.

Detection of Rolling Circle Products

The generated rolling circle amplification product can be detected by detecting a label covalently or non-covalently associated with said rolling circle amplification product, wherein said label is preferably fluorescently detectable, wherein said label is either a fluorescent molecule incorporated into the rolling circle amplification product, for example by being present in the primer used for probe amplification and generation of the rolling circle amplification product, or by being linked to a nucleotide incorporated into the rolling circle amplification product during the probe amplification process, or by being linked to a fluorescently labeled oligonucleotide hybridizing to the rolling circle amplification product, or wherein said label is a molecule or a chemical group which can be detected by a fluorescently labeled molecule, such as an antibody. Said amplification being indicative of the presence in said sample of at least one enzyme activity capable of repairing said unprocessed substrate moiety.

If detection is obtained through hybridization of a labeled oligonucleotide to the identifier elements, the identifiers need to have a certain length to be specific for a target sequence and allow hybridization under the reaction conditions. In theory an identifier could match the total length of the probe, but in most cases a shorter identifier element would be preferable. Shorter identifiers would have faster hybridization kinetics and would enable a probe to contain more than one identifier.

Thus, in one embodiment, the invention relates to an element defining the specific probe, which is a nucleotide sequence of 6-200 nucleotides, such as e.g. 6-150 nucleotides, or such as e.g. 6-100 nucleotides, or such as e.g. 6-80 nucleotides, or such as e.g. 6-60 nucleotides, or such as e.g. 6-50 nucleotides, or such as e.g. 10-40 nucleotides, or such as e.g. 10-30 nucleotides, or such as e.g. 15-30 nucleotides. However, since the probes are used as templates in rolling circle replications, detection can also be obtained through synthesis. Such detection through synthesis could be performed similar to established linear PRINS reactions. Whereas incorporation of a labeled (e.g. a flourophore) A, T, G, C, or U is an obvious approach, it will give rise to background staining, as these nucleotides could be incorporated not only in the rolling circle product but also elsewhere in the sample. Incorporating one or more artificial nucleotides, such as isoC or isoG, into the sequence of the padlock probe and providing the complementary nucleotide as a labeled nucleotide (e.g. a fluorophore) during rolling circle DNA synthesis may therefore be preferable. Since such artificial nucleotides are not found in nature, they will not be incorporated to any great extent elsewhere in the sample, minimizing background reactions. This aspect makes the use of a fluorophore-coupled isodCTP nucleotides or iso-dGTP nucleotides preferable. If detection is obtained through synthesis, the identifier element, defining the specific probe, may therefore preferably be one or more artificial nucleotide. Thus, in another embodiment, the invention relates to an element defining the specific probe, which is composed of one or more artificial nucleotides, such as e.g. 1-20 artificial nucleotides, or such as e.g. 1-10 artificial nucleotides, or such as e.g. 1-5 artificial nucleotides, or such as e.g. 4 artificial 30 nucleotides, or such as e.g. 3 artificial nucleotides, or such as e.g. 2 artificial nucleotides, or such as e.g. 1 artificial nucleotide. Thus, each probe can be identified, if desired, by e.g. primer sequence and detection sequence or both.

Washing Conditions

When the individual reactions are performed on substrates and/or padlock probes coupled to or hybridized to a solid support, it is preferable to wash the support between the individual reactions. In this way the buffer can be changed and unspecific bound cell sample debris can be removed. Several different buffers can be used. Preferably a buffer is removing most unbound sample debris without removing too much of the hybridized probe. Example of washing buffer could be, but not limited to: I) 0.1 M tris-HCl, 150 mM NaCl and 0.5% tween 20. II) 2×SSC and 0.5% tween 20 or III) 0.1 M tris-HCl, 150 mM NaCl and 0.3% SDS. Following washing the slide can either be air-dried or dehydrated through a series of ethanol (e.g. 70%, 85% and 99%) and air-dried.

Alternatively, ligated padlock probes can be detected by running a PCR which only gives a product when the padlock probe has been ligated. Subsequently the PCR product can be visualized on a gel, or, if one or more labeled nucleotides are positioned in one or both primers, the PCR product can be hybridized to an array.

Thus in one embodiment of the invention the ligated padlock probes are detected using PCR, wherein the generated PCR product optionally is hybridized to an array.

Another possibility is to use both rolling circle DNA synthesis and PCR. This can be done If the PCR primers are designed only to give a PCR product if a rolling circle reaction has taken place [14].

Thus in another embodiment of the invention the ligated padlock probes are detected using rolling circle replication, wherein part of the rolling circle product is amplified by PCR, wherein the generated PCR product optionally is hybridized to an array.

Detection of Repair of One or More Mismatched Nucleotides

In the case where the double stranded oligonucleotide probe comprises one or more mismatched nucleobases the repair of one of the strands can be monitored by hybridizing and ligating a padlock probe to one of the strands following incubation with a biological sample (perhaps) able to repair the mismatch. A padlock is able to discriminate single nucleotide variations and can thus be used to determine if the mismatch has been repaired. If the mismatch has been repaired the padlock probe can be ligated and subsequently amplified by e.g. rolling circle replication. If the mismatch has not been repaired the padlock probe can hybridize to but not be ligated since no ligation substrate has been created and thus no rolling circle product can be generated.

Alternatively, two padlock probes can be applied, one able to be ligated on the repaired substrate and one able to be ligated on the un-repaired substrate. Each padlock probe can have an identifier element enabling discrimination of each rolling circle product. In this way the degree of repair can be monitored.

Detection of Repair of One or More Loops

In the case where the double stranded oligonucleotide probe comprises one or more loops the repair of one of the strands can be monitored by hybridizing and ligating a padlock probe to one of the strands following incubation with a biological sample (perhaps) able to repair the loop structure. A padlock is able to discriminate single nucleotide variations and can thus be used to determine if the loop has been repaired. If the loop has been repaired the padlock probe can be ligated and subsequently amplified by e.g. rolling circle replication. If the loop has not been repaired the padlock probe can hybridize to (the degree of hybridization depends on the precise sequences) but not be ligated since no ligation substrate has been created and thus no rolling circle product can be generated.

Alternatively, two padlock probes can be applied, one able to be ligated on the repaired substrate and one able to be ligated on the un-repaired substrate. Each padlock probe can have an identifier element enabling discrimination of each rolling circle product. In this way the degree of repair can be monitored.

Since both repair of mismatched nucleotides and loops are known to be induced by have a nick positioned 5′ or 3′ to the damage it may be an advantage to have a nick positioned in the double stranded oligonucleotide probe. This can be done by either assembling the double stranded oligonucleotide probe from several oligonucleotides or by nicking the double stranded oligonucleotide probe with a nicking enzyme either 5′ or 3′ to the damage before applying the mixture which is going to repair the damage.

Thus in a preferred embodiment of the invention, the invention relates to a method wherein the double stranded oligonucleotide probe further comprises recognitions sites for one or more nicking enzymes.

Detection of Repair of One or More Damaged Nucleotides

In the case where the double stranded oligonucleotide probe comprises one or more damaged nucleotides the repair of one of the strands can be monitored by hybridizing and ligating a padlock probe to one of the strands following incubation with a biological sample (perhaps) able to repair the damaged nucleotide. A padlock is able to discriminate single nucleotide variations and can thus be used to determine if the damaged nucleotide has been repaired. If the one or more damaged nucleotides have been repaired the padlock probe can be ligated and subsequently amplified by e.g. rolling circle replication. If the one or more damaged nucleotides have not been repaired the padlock probe can hybridize to but not be ligated since no ligation substrate has been created and thus no rolling circle product can be generated. Alternatively, two padlock probes can be applied, one able to be ligated on the repaired substrate and one able to be ligated on the un-repaired substrate. Each padlock probe can have an identifier element enabling discrimination of each rolling circle product. In this way the degree of repair can be monitored.

In the case that the padlock probe is not able to discriminate a damaged nucleotide from a repaired nucleotide, an additional step following incubation with a mixture can be introduced. By incubating with a glycosylase (and if necessary also APE1) the damaged nucleotide can be cleaved, and thus lead to destruction of a hybridization partner for the padlock probe if the damaged nucleotide has not been repaired.

Detection of Repair of One or More Nicks

In the case where the double stranded oligonucleotide probe comprises one or more nicks, the repair of one of the strands can be monitored by hybridizing and ligating a padlock probe to one of the strands following incubation with a biological sample (perhaps) able to repair the nick. A padlock is able to discriminate single nucleotide variations and can thus be used to determine if the nick has been repaired. If the one or more nicks have been repaired the padlock probe can be ligated and subsequently amplified by e.g. rolling circle replication. If the one or more nicks have not been repaired the padlock probe can hybridize to ((the degree of hybridization depends on the precise sequences) but not be ligated since no ligation substrate has been created and thus no rolling circle product can be generated.

Alternatively, two padlock probes can be applied, one able to be ligated on the repaired substrate and one able to be ligated on the un-repaired substrate. Each padlock probe can have an identifier element enabling discrimination of each rolling circle product. In this way the degree of repair can be monitored.

Since nicks a repaired more efficient when the 5′ end of the nick is phosphorylated it can be an advantage to have 5′-phosphate positioned in the nick. Thus, in one embodiment a 5′-phosphate is positioned in the nick.

If you want to also test for kinase activity present in the sample, the 5′-phoshate in the nick can be omitted. Thus, in a second aspect of the invention a 5′-OH is present in the nick.

Detection of Repair of One or More Gaps

In the case where the double stranded oligonucleotide probe comprises one or more gaps, the repair of one of the strands can be monitored by hybridizing and ligating a padlock probe to one of the strands following incubation with a biological sample (perhaps) able to repair the gap. A padlock is able to discriminate single nucleotide variations and can thus be used to determine if the gap has been repaired. If the one or more gaps have been repaired the padlock probe can be ligated and subsequently amplified by e.g. rolling circle replication. If the one or more gaps have not been repaired the padlock probe can hybridize to (the degree of hybridization depends on the precise sequences) but not be ligated since no ligation substrate has been created and thus no rolling circle product can be generated.

Alternatively, two padlock probes can be applied, one able to be ligated on the repaired substrate and one able to be ligated on the un-repaired substrate. Each padlock probe can have an identifier element enabling discrimination of each rolling circle product. In this way the degree of repair can be monitored.

Since gaps a repaired more efficient when the 5′ end of the gap is phosphorylated it can be an advantage to have 5′-phosphate positioned in the gap. Thus, in one embodiment a 5′-phosphate is positioned in the gap.

If you want to also test for kinase activity present in the sample the 5′-phoshate in the gap can be omitted. Thus, in a second aspect of the invention a 5′-OH is present in the gap.

Detection of Repair of Overhangs

In the case where the double stranded oligonucleotide probe comprises one or more overhangs, the repair of one of the strands can be monitored by hybridizing and ligating a padlock probe to one of the strands following incubation with a biological sample (perhaps) able to repair the overhang. A padlock is able to discriminate single nucleotide variations and can thus be used to determine if the overhang has been repaired. If the one or more overhangs have been repaired the padlock probe can be ligated and subsequently amplified by e.g. rolling circle replication. If the one or more overhangs have not been repaired the padlock probe can hybridize to (the degree of hybridization depends on the precise sequences) but not be ligated since no ligation substrate has been created and thus no rolling circle product can be generated. Alternatively, two padlock probes can be applied, one able to be ligated on the repaired substrate and one able to be ligated on the un-repaired substrate. Each padlock probe can have an identifier element enabling discrimination of each rolling circle product. In this way the degree of repair can be monitored.

Self-Templating Probe for Enzyme Activity Detection:

By having a circular probe which contains a modified base, sugar group or internucleoside linker, the repair of said circular probe could be monitored. The probe could be incubated with a sample which might be able to repair the modified base, sugar group or internucleoside linker. Following sample incubation the un-repaired probes could be linearized by incubating the probe with e.g. a specific glycosylase which is able to recognize and cleave the modified base positioned in the probe. Examples of glycosylases able to recognize and cleave damaged bases are listed FIG. 12 [15] (http://www.cgal.icnet.uk/DNA Repair Genes.html). In this way unprocessed probes will be linearized, whereas repaired probes would still be circular.

In this way repaired probes could be amplified by e.g. rolling circle DNA synthesis (or PCR, as described previously), whereas un-repaired probes (and thus linear after glycosylase incubation) could not be amplified (FIGS. 7-8).

Thus in another aspect of the invention, the invention relates to a method for detecting repair of damaged nucleotides in a circular probe

Furthermore, it may be possible to use a polymerase which is unable to incorporate nucleotides across one or more of the listed damages. In that case the glycosylase step can be omitted, and the rolling circle DNA synthesis will be indicative of a repair event.

Thus, in one embodiment the invention refers to method where a polymerase unable to perform polymerization over a damaged base is used.

The probes of the invention are comprising one or more individual nucleic acid sequences. The probe can comprise any sequence of the natural nucleotides G, C, A, T, I, U, or any artificial nucleotides e.g., but not limited to, iso-dCTP, iso-dGTP, nucleotides which are substrates for glycosylases or a mixture thereof [16]. The one or more individual nucleic acid sequences of the probes of the invention have a linear length of 20-200 nucleotides. Thus, in one aspect, the invention relates to a method, wherein the one or more probes have a length of 20-300 nucleotides, such as e.g. 20-150 nucleotides, or such as e.g. 20-100 nucleotides, or such as e.g. 20-80 nucleotides, or such as e.g. 20-60 nucleotides, or such as e.g. 20-40 nucleotides, or such as e.g. 20-30 nucleotides. The probes can be synthesized by standard chemical methods (e.g. beta-cyanoethyl phosphoramidite chemistry).

The probes possess several characteristics: 1) the probe comprises one or more complementary sequences, enabling the probe to hybridize to itself. 2) the probe comprises one or more loop structures connecting complementary sequences. 3) The probe possesses binding or interaction sites for one or more enzymes (so-called substrate moieties). 4) The probes are designed so that no part of the probe recognizes DNA or RNA sequences in the sample. See also FIGS. 7-8.

Loop Structure of the Probes

The one or more loop structures of the probe aim to connect the ends of the two or more complementary sequences. The loop comprises 3-100 nucleotides, such as e.g. 3-80 nucleotides, or such as e.g. 3-60 nucleotides, or such as e.g. 3-40 nucleotides, or such as e.g. 3-30 nucleotides. The loop structures can serve one or more purposes. The loop can be used as primer recognition sequences for amplification reactions, e.g. for rolling circle DNA synthesis, or PCR. The loops can also serve as an identification element to identify specific probes. The loops also serve to connect one or more double stranded regions of the probe.

Complementary Sequences of the Probes

The complementary sequences of the probe are positioned on each side of the one or more loop structures in the sequence of the probe. The complementary sequences comprise 3-100 nucleotides, such as e.g. 5-80 nucleotides, such as e.g. 10-60 nucleotides, such as e.g. 10-30 nucleotides, or such as e.g. 10-40 nucleotides. Preferably, the complementary sequences are 10-20 nucleotides long, such as e.g. 10-20 nucleotides, such as e.g. 12-20 nucleotides, such as e.g. 14-20 nucleotides, or such as e.g. 15-20 nucleotides.

The aim of the complementary sequences of the probe is to form a substrate or part of a substrate for one or more enzymes. Furthermore, the complementary sequences enable the probe to be circularized by self-templated hybridization of the complementary sequences in the probe.

The probe can be provided in several formats: In one format the probe, which consists of a single oligonucleotide, through self-templated hybridization is able to form a region which can be a substrate moiety for one or more enzymes. In another format the probe, which consists of more than one oligonucleotide, through hybridization, is able to form a region which can be a substrate moiety for one or more enzymes. It is to be understood that all of the following sections refers to both formats of the probe.

Primer Design

In general, a primer consists of 5-50 nucleotides and preferably of 7-15 nucleotides. The primer has to be complementary to part of the nucleic acid probe, preferably a part outside the double stranded region. Preferably the primer is 100% complementary to the probe, alternatively nucleotides at the 5′-end of the primer are non-complementary to the probe, e.g. 1 nucleotide, 3 nucleotides, 5 nucleotides, 10 nucleotides, 25 nucleotides or 50 nucleotides. If a polymerase containing 3′ to 5′ exonuclease activity is used (e.g. Phi29 DNA polymerase), non-complementary nucleotides at the 3′-end of the primer can be present, such as e.g. 1 nucleotide, such as e.g. 3 nucleotides, such as e.g. 5 nucleotides, such as e.g. 10 nucleotides, such as e.g. 25 nucleotides, or such as e.g. 50 nucleotides. Furthermore, mismatched nucleotides in the primer can be present, e.g. 1 nucleotide, such as e.g. 3 nucleotides, such as e.g. 5 nucleotides, such as e.g. 10 nucleotides, or such as e.g. 25 nucleotides. In the case where the probe consists of more than one unbroken chain of nucleotides, one or more of the chains of nucleotides can be used as the primer.

The primer can be synthesized by standard chemical methods (e.g. beta-cyanoethyl phosphoramidite chemistry). A primer can also contain modifications e.g., but not limited to, streptavidine, avidin, biotin, ³²P, and fluorophores, amins or it may comprise artificial nucleotides such as, but not limited to, LNA, PNA, iso-dCTP, and iso-dGTP. For correct annealing between circle and primer, a molar ratio of 0.1-100 between circle and primer is mixed, preferably 0.8-1.2.

It is to be understood that polymerases which do not need a primer can also be used by the method of the invention. In this case no primers are needed to start the rolling circle replication.

The primers, in the method of the invention, can be anchored to a solid support, thereby attaching the following rolling circle product to a surface. This will make it easier to change buffer conditions, and improve washing between the different steps, thereby minimizing background. In one example, the primer can be coupled in the 5′-end to a solid support—a 5′-biotin labeled primer may e.g. be coupled to a streptavidine coated solid support including, but not limited to, PCR-tubes, ELISA plates, beads, plastic CDs (e.g. produced by the company Åmic), and microscope slides. In another example the primer is coupled to a solid support through a 5′-amin, thereby getting a covalent linkage, including, but not limited to, PCR-tubes, ELISA plates, beads, plastic CDs (produced by the company Åmic), and microscope slides. It is to be understood that the primer can also be coupled to a surface when it is part of the probe. Thus, in one aspect, the invention relates to a method, wherein the primer is immobilized on a solid support.

Probe Hybridization to Primer

The primer can already be present in the sample incubation step, but preferably the primer is added subsequently to sample incubation. The primer can be added together with the polymerase or in an individual hybridization step prior to rolling circle DNA synthesis. If the primer is linked to a solid support, the mixture can be supplemented with 0.01-2 M NaCl (final concentration) to increase hybridization; preferably the mixture is supplemented with 500 mM NaCl (final concentration). If the primer is linked to a solid support, protease digestion can also be performed following probe hybridization to the primer, thereby removing protein cell debris from the solid support.

Washing Conditions

If the primer is coupled to a solid support and the probe is hybridized to the primer, it is preferable to wash the support before initiation of rolling circle DNA synthesis. In this way the buffer can be changed and unspecific bound cell sample debris can be removed. Several different buffers can be used. Preferably a buffer is removing most unbound sample debris without removing too much of the hybridized probe. Example of washing buffer could be, but not limited to: I) 0.1 M tris-HCl, 150 mM NaCl and 0.5% tween 20. II) 2×SSC and 0.5% tween 20 or III) 0.1 M tris-HCl, 150 mM NaCl and 0.3% SDS. Following washing the slide can either be air-dried or dehydrated through a series of ethanol (e.g. 70%, 85% and 99%) and air-dried.

Alternatively the repair event can be detected by PCR by positioning a primer in each site of the gap.

In the Case of Repair of One or More Damaged Nucleotides

In the case that the probe has been repaired, no substrate for a glycosylase is present in the probe. This means that the probe cannot be linearized by a glycosylase, and the circular probe is substrate for a rolling circle replication reaction.

Examples of oxidized nucleotides are given in FIG. 10, and an overview of where damages occurs in nucleotides is given in FIG. 11.

In the Case of No Repair of One or More Damaged Nucleotides

In the case that the probe has not been repaired, a substrate for a glycosylase is present in the probe. This means that the probe can be linearized by a glycosylase, and thus the linearized probe is no longer substrate for a rolling circle replication reaction.

Thus in one embodiment of the invention, a rolling circle product is indicative of the presence in the biological sample of a nucleotide repair activity involved in repairing a damaged nucleotide in a circular oligonucleotide strand, and no amplification product is formed in the absence of such enzyme activity.

Reference is made to the below-cited disclosures which are hereby incorporated herein by reference in their entirety.
1. Lisby M, Olesen J R, Skouboe C, Krogh B O, Straub T, Boege F, Velmurugan S, Martensen P M, Andersen A H, Jayaram M et al: Residues within the N-terminal domain of human topoisomerase I play a direct role in relaxation. J Biol Chem 2001, 276(23):20220-20227.
2. Friedrich-Heineken E, Hubscher U: The Fen1 extrahelical 3′-flap pocket is conserved from archaea to human and regulates DNA substrate specificity. Nucleic Acids Res 2004, 32(8):2520-2528.
3. Collins A R: The comet assay for DNA damage and repair: principles, applications, and limitations. Mol Biotechnol 2004, 26(3):249-261.
4. Lindahl T: Instability and decay of the primary structure of DNA. Nature 1993, 362(6422):709-715.
5. Barnes D E, Lindahl T: Repair and genetic consequences of endogenous DNA base damage in mammalian cells. Annu Rev Genet 2004, 38:445-476.
6. Ames B N, Shigenaga M K, Hagen T M: Oxidants, antioxidants, and the degenerative diseases of aging. Proc Natl Acad Sci USA 1993, 90(17):7915-7922.
7. Sedgwick B, Bates P A, Paik J, Jacobs S C, Lindahl T: Repair of alkylated DNA: recent advances. DNA Repair (Amst) 2007, 6(4):429-442.
8. Spry M, Scott T, Pierce H, D'Orazio J A: DNA repair pathways and hereditary cancer susceptibility syndromes. Front Biosci 2007, 12:4191-4207.
9. Garinis G A, Jans J, van der Horst G T: Photolyases: capturing the light to battle skin cancer. Future Oncol 2006, 2(2):191-199.
10. Ide H, Kotera M: Human DNA glycosylases involved in the repair of oxidatively damaged DNA. Biol Pharm Bull 2004, 27(4):480-485.
11. Dizdaroglu M: Base-excision repair of oxidative DNA damage by DNA glycosylases. Mutat Res 2005, 591(1-2):45-59.
12. Liu Y, Kao H I, Bambara R A: Flap endonuclease 1: a central component of DNA metabolism. Annu Rev Biochem 2004, 73:589-615.
13. Iwai S: Chemical synthesis of oligonucleotides containing damaged bases for biological studies. Nucleosides Nucleotides Nucleic Acids 2006, 25(4-6):561-582.
14. Baner J, Gyarmati P, Yacoub A, Hakhverdyan M, Stenberg J, Ericsson O, Nilsson M, Landegren U, Belak S: Microarray-based molecular detection of foot-and-mouth disease, vesicular stomatitis and swine vesicular disease viruses, using padlock probes. J Virol Methods 2007.
15. Wood R D, Mitchell M, Lindahl T: Human DNA repair genes, 2005. Mutat Res 2005, 577(1-2):275-283.
16. Huffman J L, Sundheim O, Tainer J A: DNA base damage recognition and removal: new twists and grooves. Mutat Res 2005, 577(1-2):55-76.

### Examples

### Example 1

Detection of mismatch repair: A double stranded oligonucleotide probe, containing a single A-G mismatch, is covalently coupled to a solid support through a 5′-amin in one of the strands. The probe is incubated with a cell preparation for 30 min. and subsequently the cell preparation is washed away. The double stranded oligonucleotide probe is denatured through heating for 5 min at 95 C leaving only the covalently coupled strand. A padlock probe able to hybridize to and ligate on the coupled strand, if the strand has been repaired at the mismatch, is incubated with the coupled strand in the presence of T4 DNA ligase and ATP and high salt (250 mM). A rolling circle amplification is started by incubating the hybridized and ligated padlock probe with phi29 DNA polymerase and dNTPs for 30 min. The rolling circle product is visualized by hybridizing fluorescently labeled nucleotides to the rolling circle product and visualize it under the microscope. Detection of rolling circle products is indicative of the repair of the mismatched nucleotide in the coupled strand of the double stranded oligonucleotide probe. See also FIG. 1.

### Example 2

Like example 1, but with the difference that the double stranded oligonucleotide is one oligonucleotide which through self-templated hybridization is able to constitute a double stranded substrate. See also FIG. 1.

### Example 3

Like examples 1 or 2, but with the difference that a restriction digestion is performed following incubation with the cell preparation. The restriction digestion results in the appearance of a 3′-end close to where the padlock probe is hybridized and ligated. This additional step improves the rolling circle amplification since no 3′-exonuclease activity has to present. See also FIG. 1.

### Example 4

Like examples 1, 2 or 3, but with the difference that a nick is positioned 3′ to the nucleotide which is going to be repaired in the double stranded region of the probe. A nick can induce the repair event. See also FIG. 2.

### Example 5

Like examples 1, 2 or 3, but with the difference that a nick is positioned 5′ to the nucleotide which is going to be repaired in the double stranded region of the probe. A nick can induce the repair event. See also FIG. 3.

### Example 6

Detection of nick repair: A double stranded oligonucleotide probe is coupled to a solid support through a 5′-amin in one of the strands; the coupled strand comprises a nick. The probe is incubated with a cell preparation for 30 min. and subsequently the cell preparation is washed away. The double stranded oligonucleotide probe is denatured through heating for 5 min at 95 C leaving only the covalently coupled strand. A padlock probe able to hybridize to and ligate on the coupled strand, if the strand has been repaired at the nick, is incubated with the coupled strand in the presence of T4 DNA ligase and ATP and high salt (250 mM). Following a wash, a rolling circle amplification is started by incubating the hybridized and ligated padlock probe with phi29 DNA polymerase and dNTPs for 30 min. The rolling circle product is visualized by hybridizing fluorescently labeled nucleotides to the rolling circle product and visualize it under the microscope. Detection of rolling circle products is indicative of the repair of the nick in the coupled strand of the double stranded oligonucleotide probe. See also FIG. 4.

### Example 7

Like example 6, but with the difference that the double stranded oligonucleotide is one oligonucleotide which through self-templated hybridization is able to constitute a double stranded substrate comprising a nick. See also FIG. 4.

### Example 8

Like examples 6 or 7, but with the difference that a restriction digestion is performed following incubation with the cell preparation. The restriction digestion results in the appearance of a 3′-end close to where the padlock probe is hybridized and ligated.

This additional step improves the rolling circle amplification since no 3′-exonuclease activity has to present. See also FIG. 4.

### Example 9

Detection of repair of an abasic site: A self-templated circular oligonucleotide probe comprising an abasic site in the double stranded region is incubated with a cell preparation. Following incubation the probe is incubated with APE1 which will cleave the un-repaired abasic sites. Subsequently the probe is incubated with a primer (in solution or coupled to a support), and a rolling circle amplification is initiated and detected as described above. Only repaired circles will be amplified by rolling circle amplification. See also FIG. 5.

### Example 10

Like example 9, with the difference that the probe is not a self-templated probe, but composed of two oligonucleotides (a circular and a linear). The linear oligonucleotide can function as the primer. See also FIG. 6.

### Example 11

Detection of repair of 8-oxoG: A self-templated circular oligonucleotide probe comprising a 8-oxoG in the double stranded region is incubated with a cell preparation. Following incubation the probe is incubated with OGG1 which will cleave the un-repaired 8-oxoG nucleotide. Subsequently the probe is incubated with a primer (in solution or coupled to a support), and a rolling circle amplification is initiated and detected as described above. Only repaired circles will be amplified by rolling circle amplification. See also FIG. 5.

### Example 12

Like example 11, with the difference that the probe is not a self-templated probe, but composed of two oligonucleotides (a circular and a linear). The linear oligonucleotide can function as the primer. See also FIG. 6.

### Example 13

Detection of repair of uracil in DNA: Like example 11 or 12, but with the difference that the damaged nucleotide is a U and the used glycosylase is uracil-DNA-glycosylase (UDG). Following UDG incubation, a enzymatic step with APE1 can optionally be introduced to further process the probe. See also FIGS. 5 and 6.

1. A method for determining in a biological sample either
a) the presence of one or more enzyme activities involved in processing in a double stranded oligonucleotide probe one or more of
i) one or more mismatched nucleobase hybridisation event(s) in the double stranded oligonucleotide probe, and/or
ii) absence of nucleobase hybridisation(s) resulting in one or more loop formation(s) in the double stranded oligonucleotide probe, and/or
iii) presence of one or more damaged nucleotide(s) in the double stranded oligonucleotide probe, and/or
iv) one or more nick(s) in one or more of the strand(s) of the double stranded nucleotide probe, and/or
v) one or more single stranded nucleotide sequence(s) joined at one or both ends thereof by a double stranded nucleotide sequence, wherein said single stranded sequence(s) create one or more gap structure(s) in the double stranded oligonucleotide probe, and/or
vi) one or more nick(s) or one or more gap(s), wherein said gap(s) are in the form of a single stranded nucleotide sequence, wherein said nick(s) or gap(s) are joined at one end thereof to a double stranded nucleotide sequence and at the other end thereof to at least one single stranded overhang joined to a double stranded nucleotide sequence of the double stranded nucleotide probe,
or
b) the absence of at least one such enzyme activity in said biological sample,
said method comprising the steps of
i) providing a biological sample to be analysed for the presence or absence of at least one enzyme activity,
ii) providing a double stranded oligonucleotide probe comprising an unprocessed substrate moiety capable of being processed by at least one of said one or more enzyme activities,
wherein said unprocessed substrate moiety is selected from the group consisting of
i) an unprocessed substrate moiety comprising one or more mismatched nucleobase hybridisation event(s) in the double stranded oligonucleotide probe,
ii) an unprocessed substrate moiety comprising one or more loop formation(s) resulting from absence of nucleobase hybridisation(s) in the double stranded oligonucleotide probe,
iii) an unprocessed substrate moiety comprising one or more damaged nucleotide(s) in the double stranded oligonucleotide probe,
iv) an unprocessed substrate moiety comprising one or more nick(s) in one or more of the strand(s) of the double stranded nucleotide probe,
v) an unprocessed substrate moiety comprising one or more single stranded nucleotide sequence(s) joined at one or both ends thereof by a double stranded nucleotide sequence, wherein said single stranded sequence(s) create one or more gap structure(s) in the double stranded oligonucleotide probe, and
vi) an unprocessed substrate moiety comprising one or more nick(s) or one or more gap(s), wherein said gap(s) are in the form of a single stranded nucleotide sequence, wherein said nick(s) or gap(s) are joined at one end thereof to a double stranded nucleotide sequence and at the other end thereof to at least one single stranded overhang joined to a double stranded nucleotide sequence of the double stranded nucleotide probe,
wherein said double stranded oligonucleotide probe comprises a single strand of contiguous nucleotides or a plurality of single strands of contiguous nucleotides capable of hybridisation to each other,
iii) contacting the biological sample with the double stranded oligonucleotide probe under conditions allowing said one or more enzyme activities, if present in said biological sample, to act on the unprocessed substrate moiety,
wherein said action results in the processing of the unprocessed substrate moiety and the generation of a processed, double stranded oligonucleotide probe,
wherein, when said one or more enzyme activities are not present in said biological sample, no processing of the unprocessed substrate moiety takes place,
iv) separating the individual strands of the double stranded oligonucleotide probe,
v) providing a padlock probe capable of hybridising to an individual strand of the double stranded oligonucleotide probe,
wherein, when the individual strand of the double stranded oligonucleotide probe comprises an unprocessed substrate moiety, the padlock either cannot hybridise to said individual strand comprising an unprocessed substrate moiety, or, when hybridised to said individual strand comprising an unprocessed substrate moiety, the individual strand comprising an unprocessed substrate moiety does not constitute a template for ligation of the nucleotide ends of the padlock probe, in which case the padlock probe cannot be ligated by a ligase and serve as a circular template for rolling circle replication,
wherein, when the individual strand comprises a processed substrate moiety, the padlock probe is hybridised to said individual strand and ligated by a ligase, thereby providing a circular template for rolling circle replication,
vi) providing a ligase capable of ligating the ends of the padlock probe, thereby generating a circular template for rolling circle replication,
vii) selectively ligating padlock probes hybridised to an individual strand comprising a processed substrate moiety,
wherein said padlock probe ligation results in the formation of a circular oligonucleotide template capable of being amplified by rolling circle replication,
viii) amplifying the circular oligonucleotide template generated in step vii), by using a polymerase capable of performing multiple rounds of rolling circle replication of said circular oligonucleotide template, optionally by contacting said circular oligonucleotide template with a suitable primer, and generating a rolling circle amplification product comprising multiple copies of the circular oligonucleotide template, or
ix) generating no rolling circle amplification product when no padlock probe ligation takes place,
wherein steps viii) and ix) are mutually exclusive,
wherein said amplification product is indicative of the presence in said biological sample of said one or more enzyme activities involved in processing, in a double stranded oligonucleotide probe, one or more of
i) one or more mismatched nucleobase hybridisation event(s) in the double stranded oligonucleotide probe, and/or
ii) absence of nucleobase hybridisation(s) resulting in one or more loop formation(s) in the double stranded oligonucleotide probe, and/or
iii) presence of one or more damaged nucleotide(s) in the double stranded oligonucleotide probe, and/or
iv) one or more nick(s) in one or more of the strand(s) of the double stranded nucleotide probe, and/or
v) one or more single stranded nucleotide sequence(s) joined at one or both ends thereof by a double stranded nucleotide sequence, wherein said single stranded sequence(s) create one or more gap structure(s) in the double stranded oligonucleotide probe, and/or
vi) one or more nick(s) or one or more gap(s), wherein said gap(s) are in the form of a single stranded nucleotide sequence, wherein said nick(s) or gap(s) are joined at one end thereof to a double stranded nucleotide sequence and at the other end thereof to at least one single stranded overhang joined to a double stranded nucleotide sequence of the double stranded nucleotide probe,
and
wherein no rolling circle amplification product is formed in the absence of such an enzyme activity.

2. A method for determining in a biological sample either
a) the presence of one or more nucleotide repair enzyme activities involved in repairing a damaged nucleotide in a circular oligonucleotide probe, or
b) the absence of such a nucleotide repair enzyme activity,
said method comprising the steps of
i) providing a biological sample to be analysed for the presence or absence of a nucleotide repair enzyme activity involved in repairing a damaged nucleotide,
ii) providing an oligonucleotide probe comprising one or more strands,
wherein at least one of said one or more strands is in the form of a circular oligonucleotide comprising a damaged nucleotide capable of being repaired by the nucleotide repair enzyme activity,
wherein said oligonucleotide probe is selected from the group consisting of a circular, self-templated oligonucleotide probe comprising a single strand of contiguous nucleotides capable of hybridising to a primer for priming rolling circle amplification of said circular, self-templated oligonucleotide probe and a circular oligonucleotide probe comprising a plurality of single strands of contiguous nucleotides capable of hybridisation to each other, wherein at least one of said single strands is in the form of a circular oligonucleotide, wherein at least one of the plurality of single strands is capable of hybridising to the circular oligonucleotide and priming rolling circle amplification of said circular oligonucleotide,
iii) incubating the biological sample and the circular oligonucleotide probe comprising a damaged nucleotide capable of being repaired by the nucleotide repair enzyme activity under conditions allowing said nucleotide repair enzyme activity, if present in said biological sample, to act on the damaged nucleotide,
wherein said action results in repairing the damaged nucleotide, and
wherein no repair of the damaged nucleotide occurs in the absence of said one or more nucleotide repair enzyme activities in said biological sample,
iv) providing a glycosylase enzyme activity and contacting the glycosylase enzyme activity with the self-templating circular oligonucleotide probe provided in step ii) after said probe has been incubated with the biological sample,
wherein the glycosylase enzyme activity cleaves the circular oligonucleotide strand having an unrepaired and damaged nucleotide, thereby generating a linear oligonucleotide which cannot be amplified by rolling circle amplification, and
wherein the glycosylase enzyme activity does not cleave the circular oligonucleotide strand having a repaired nucleotide, wherein said circular oligonucleotide strand can be amplified by rolling circle replication,
v) priming the circular oligonucleotide strand having been contacted with the glycosylase enzyme activity, optionally by providing a primer, and
vi) amplifying the circular oligonucleotide strand comprising a repaired nucleotide, when such a circular oligonucleotide strand is formed in step iv), by using a polymerase capable of performing multiple rounds of rolling circle replication of said circular oligonucleotide strand, and generating a rolling circle amplification product comprising multiple copies of the circular oligonucleotide strand, or
viii) generating no rolling circle amplification product when no circular oligonucleotide strand is formed in step iv) as a result of said glycosylase having excised said damaged nucleotide from said circular oligonucleotide strand and thereby having generated a linear oligonucleotide strand,
wherein steps vi) and vii) are mutually exclusive,
wherein said amplification product is indicative of the presence in said biological sample of a nucleotide repair enzyme activity involved in repairing a damaged nucleotide in a circular oligonucleotide strand
and
wherein no amplification product is formed in the absence of such an enzyme activity.

3-9. (canceled)

10. The method of claim 1, wherein the unprocessed substrate moiety comprises a nick or a single stranded nucleotide region.

11. The method of claim 10, wherein the single stranded nucleotide region is adjoined at both ends to a double stranded nucleotide region.

12-18. (canceled)

19. The method of claim 11, wherein the substrate moiety conversion is mediated specifically by a flap endonuclease activity present in said sample in combination with a ligase activity present in said sample and/or added to said sample.

20. The method of claim 19, wherein the flap endonuclease activity is mediated by FEN1, DNA2P or EXO1.

21. The method of claim 11, wherein the substrate moiety conversion is mediated specifically by a topoisomerase activity present in said sample.

22. The method of claim 21, wherein the topoisomerase activity is mediated by a Topoisomerase I or Topoisomerase II.

23. (canceled)

24. The method of claim 1, wherein said unprocessed substrate moiety is selected from the group consisting of
i) unprocessed substrate moieties comprising or consisting of one or more nick(s) in one or more single strand(s) of a double stranded nucleotide sequence of said oligonucleotide probe, said one or more nick(s) forming one or more unprocessed substrate moieties of said oligonucleotide probe,
ii) unprocessed substrate moieties comprising or consisting of one or more single stranded nucleotide sequence(s) joined at one or both ends thereof by a double stranded nucleotide sequence, said single stranded sequence(s) creating one or more gap structure(s) forming one or more unprocessed substrate moieties of said oligonucleotide probe, and
iii) unprocessed substrate moieties comprising or consisting of one or more nick(s) or one or more gap(s), said gap(s) being in the form of a single stranded nucleotide sequence, said nick(s) or gap(s) being joined at one end thereof to a double stranded nucleotide sequence and at the other end thereof to at least one single stranded overhang joined to a double stranded nucleotide sequence of said oligonucleotide probe, wherein said nick(s) or gap(s) in combination with the at least one single stranded overhang forms one or more unprocessed substrate moieties of said oligonucleotide probe.

25-34. (canceled)

35. The method of claim 24, wherein the one or more overhang(s) is a 5′ overhang, said oligonucleotide probe further comprising at least one 3′ end.

36. The method of claim 35, wherein the 5′ overhang is protected by a protection group preventing an exonuclease from digesting the 5′ overhang.

37-51. (canceled)

52. The method of claim 35, wherein the nucleotides of the 5′ overhang each comprise a nucleobase and a backbone unit, wherein the backbone unit comprises a sugar moiety and an internucleoside linker.

53. The method of claim 52, wherein the nucleobase of the nucleotides of the 5′ overhang are selected from naturally occurring nucleobases and non-naturally occurring nucleobases.

54-55. (canceled)

56. The method of claim 52, wherein the internucleoside linker of the backbone unit of neighbouring nucleobases is selected from naturally occurring internucleoside linkers and non-naturally occurring internucleoside linkers.

57. (canceled)

58. The method of claim 52, wherein the nucleobases of the 5′ overhang are selected independently from the group consisting of adenine, guanine, isoguanine, thymine, cytosine, isocytosine, pseudoisocytosine, uracil, inosine, purine, xanthine, diaminopurine, 8-oxo-N⁶-methyladenine, 7-deazaxanthine, 7-deazaguanine, N⁴,N⁴-ethanocytosin, N⁶,N⁶-ethano-2,6-diamino-purine, 5-methylcytosine, 5-(C³-C⁶)-alkynylcytosine, 5-fluorouracil, 5-bromouracil and 2-hydroxy-5-methyl-4-triazolopyridine.

59-72. (canceled)

73. The method of claim 35, wherein the nucleotides of the 5′ overhang are selected from naturally occurring nucleosides of the DNA and RNA family connected through phosphodiester linkages and at least one non-natural nucleotide selected from the group consisting of nucleotides comprising a non-natural nucleobase, nucleotides comprising a non-natural backbone unit comprising a non-natural sugar moiety, and nucleotides comprising a non-natural internucleoside linker.

74-88. (canceled)

89. The method of claim 53, wherein said 5′ overhang comprises non-naturally occurring nucleobases connected by naturally occurring backbone units and/or non-naturally occurring backbone units, said backbone units comprising a sugar moiety and an internucleoside linker, wherein said non-naturally occurring nucleobases and said non-naturally occurring backbone units, when present, prevent exonuclease degradation of said 5′ overhang.

90-98. (canceled)

99. The method of claim 34, wherein the one or more overhang(s) is a 3′ overhang, said oligonucleotide probe further comprising at least one 5′ end.

100. The method of claim 99, wherein the 3′ overhang is protected by a protection group preventing an exonuclease from digesting the 3′ overhang.

101. The method of claim 100, wherein a hybridisation partner for padlock probe hybridisation and ligation is generated by a) endonucleolytic digestion of said 3′ overhang and b) ligation of the end of the nucleotide strand resulting from the endonucleolytic digestion to a nucleotide strand of the remaining part of the oligonucleotide probe.

102. The method of claim 101, wherein said padlock probe is hybridised to the hybridisation partner and ligated and amplified by rolling circle amplification, said amplification being indicative of the presence in said sample of at least one endonuclease.

103. The method of claim 102, wherein said rolling circle amplification product is detected by detecting a label covalently or non-covalently associated with said rolling circle amplification product.

104-107. (canceled)

108. The method of claim 99, wherein the nucleotides of the 3′ overhang each comprises a nucleobase and a backbone unit, wherein the backbone unit comprises a sugar moiety and an internucleoside linker.

109. The method of claim 108, wherein the nucleobase of the nucleotides of the 3′ overhang are selected from naturally occurring nucleobases and non-naturally occurring nucleobases.

110-128. (canceled)

129. The method of claim 99, wherein the nucleotides of the 3′ overhang are selected from naturally occurring nucleosides of the DNA and RNA family connected through phosphodiester linkages and at least one non-natural nucleotide selected from the group consisting of nucleotides comprising a non-natural nucleobase, nucleotides comprising a non-natural backbone unit comprising a non-natural sugar moiety, and nucleotides comprising a non-natural internucleoside linker.

130-135. (canceled)

136. The method of claim 109, wherein said 3′ overhang comprises naturally occurring nucleobases connected by naturally occurring backbone units, wherein said naturally occurring nucleobases and said naturally occurring backbone units do not prevent exonuclease degradation of said 3′ overhang.

137-144. (canceled)

145. The method of claim 109, wherein said 3′ overhang comprises non-naturally occurring nucleobases connected by naturally occurring backbone units and/or non-naturally occurring backbone units, said backbone units comprising a sugar moiety and an internucleoside linker, wherein said non-naturally occurring nucleobases and said non-naturally occurring backbone units, when present, prevent exonuclease degradation of said 3′ overhang.

146-154. (canceled)

155. The method of claim 1, wherein the double stranded region further comprises one or more recognition sites for one or more nicking enzymes.

156. (canceled)

157. The method of claim 1, wherein the oligonucleotide probe is coupled covalently or non-covalently to a solid support.

158-160. (canceled)

161. The method of claim 1, wherein the one or more damaged nucleotides are selected from the group consisting of nucleotides damaged by oxidation, deamination and alkylation.

162-170. (canceled)

171. A liquid composition comprising
a) one or more oligonucleotide probes selected from the group consisting of
i) oligonucleotide probes comprising unprocessed substrate moieties comprising one or more nick(s) in one or more single strand(s) of a double stranded nucleotide sequence of said oligonucleotide probe, said one or more nick(s) forming one or more unprocessed substrate moieties of said oligonucleotide probe,
ii) oligonucleotide probes comprising unprocessed substrate moieties comprising one or more single stranded nucleotide sequence(s) joined at one or both ends thereof by a double stranded nucleotide sequence, said single stranded sequence(s) creating one or more gap structure(s) forming one or more unprocessed substrate moieties of said oligonucleotide probe,
iii) oligonucleotide probes comprising unprocessed substrate moieties comprising one or more nick(s) or one or more gap(s), said gap(s) being in the form of a single stranded nucleotide sequence, said nick(s) or gap(s) being joined at one end thereof to a double stranded nucleotide sequence and at the other end thereof to at least one single stranded overhang joined to a double stranded nucleotide sequence of said oligonucleotide probe, wherein said nick(s) or gap(s) in combination with the at least one single stranded overhang forms one or more unprocessed substrate moieties of said oligonucleotide probe;
iv) oligonucleotide probes comprising unprocessed substrate moieties comprising one or more mismatched nucleobase hybridisation event(s) in the double stranded oligonucleotide probe,
v) oligonucleotide probes comprising unprocessed substrate moieties comprising the absence of nucleobase hybridisation(s) resulting in one or more loop formation(s) in the double stranded oligonucleotide probe, and
vi) oligonucleotide probes comprising unprocessed substrate moieties comprising one or more damaged nucleotide(s) in the double stranded oligonucleotide probe,
and
b) a liquid carrier allowing one or more enzymes to process the one or more unprocessed substrate moieties of said one or more oligonucleotide probes.

172. A composition comprising a tissue sample, or a biopsy sample, obtained from an animal, and the liquid composition according to claim 171.

173. A solid support coupled to the oligonucleotide probe as defined in claim 1.

174. A solid support comprising a plurality of attachment points for the attachment to the solid support of one or more oligonucleotide probes each comprising one or more unprocessed substrate moieties, wherein an oligonucleotide probe is either directly attached to an attachment point through one strand of the oligonucleotide probe, wherein said strand is capable of initiating rolling circle amplification of a second strand of the oligonucleotide probe, or an oligonucleotide probe is attached to an attachment point through hybridisation of the oligonucleotide probe to a primer oligonucleotide attached to an attachment point, wherein said primer is capable of initiating rolling circle amplification of the oligonucleotide probe, so that individual attachment points are associated with one or more oligonucleotide primers suitable for initiating rolling circle amplification of a circular template generated by enzyme processing of said one or more oligonucleotide probes each comprising one or more unprocessed substrate moieties,
wherein the same or different primers are associated with the same or different attachment points,
wherein the oligonucleotide probes attached to the solid support are selected from the group consisting of
i) oligonucleotide probes comprising unprocessed substrate moieties comprising one or more nick(s) in one or more single strand(s) of a double stranded nucleotide sequence of said oligonucleotide probe, said one or more nick(s) forming one or more unprocessed substrate moieties of said oligonucleotide probe,
ii) oligonucleotide probes comprising unprocessed substrate moieties comprising one or more single stranded nucleotide sequence(s) joined at one or both ends thereof by a double stranded nucleotide sequence, said single stranded sequence(s) creating one or more gap structure(s) forming one or more unprocessed substrate moieties of said oligonucleotide probe,
iii) oligonucleotide probes comprising unprocessed substrate moieties comprising one or more nick(s) or one or more gap(s), said gap(s) being in the form of a single stranded nucleotide sequence, said nick(s) or gap(s) being joined at one end thereof to a double stranded nucleotide sequence and at the other end thereof to at least one single stranded overhang joined to a double stranded nucleotide sequence of said oligonucleotide probe, wherein said nick(s) or gap(s) in combination with the at least one single stranded overhang forms one or more unprocessed substrate moieties of said oligonucleotide probe,
iv) oligonucleotide probes comprising unprocessed substrate moieties comprising one or more mismatched nucleobase hybridisation event(s) in the double stranded oligonucleotide probe,
v) oligonucleotide probes comprising unprocessed substrate moieties comprising the absence of nucleobase hybridisation(s) resulting in one or more loop formation(s) in the double stranded oligonucleotide probe, and
vi) oligonucleotide probes comprising unprocessed substrate moieties comprising one or more damaged nucleotide(s) in the double stranded oligonucleotide probe.

175-176. (canceled)

177. A microfluidic device comprising one or more reaction compartments for performing one or more rolling circle amplification events of a circular oligonucleotide template and one or more detection compartments for the detection of said rolling circle amplification events performed in said one or more reaction compartments.

178. (canceled)

179. A method for correlating one or more rolling circle amplification event(s) with the activity of one or more enzymes in a sample, said method comprising the steps of performing the method according to claim 1 and amplifying by rolling circle amplification the one or more circular templates having been generated as a result of the presence in said sample of said one or more enzyme activities, wherein the detection of said amplification events is done using
a solid support comprising a plurality of attachment points for the attachment to the solid support of one or more oligonucleotide probes each comprising one or more unprocessed substrate moieties, wherein an oligonucleotide probe is either directly attached to an attachment point through one strand of the oligonucleotide probe, wherein said strand is capable of initiating rolling circle amplification of a second strand of the oligonucleotide probe, or an oligonucleotide probe is attached to an attachment point through hybridisation of the oligonucleotide probe to a primer oligonucleotide attached to an attachment point, wherein said primer is capable of initiating rolling circle amplification of the oligonucleotide probe, so that individual attachment points are associated with one or more oligonucleotide primers suitable for initiating rolling circle amplification of a circular template generated by enzyme processing of said one or more oligonucleotide probes each comprising one or more unprocessed substrate moieties,
wherein the same or different primers are associated with the same or different attachment points, wherein the oligonucleotide probes attached to the solid support are selected from the group consisting of
a) oligonucleotide probes comprising unprocessed substrate moieties comprising one or more nick(s) in one or more single strand(s) of a double stranded nucleotide sequence of said oligonucleotide probe, said one or more nick(s) forming one or more unprocessed substrate moieties of said oligonucleotide probe,
b) oligonucleotide probes comprising unprocessed substrate moieties comprising one or more single stranded nucleotide sequence(s) joined at one or both ends thereof by a double stranded nucleotide sequence, said single stranded sequence(s) creating one or more gap structure(s) forming one or more unprocessed substrate moieties of said oligonucleotide probe,
c) oligonucleotide probes comprising unprocessed substrate moieties comprising one or more nick(s) or one or more gap(s), said gap(s) being in the form of a single stranded nucleotide sequence, said nick(s) or gap(s) being joined at one end thereof to a double stranded nucleotide sequence and at the other end thereof to at least one single stranded overhang joined to a double stranded nucleotide sequence of said oligonucleotide probe, wherein said nick(s) or gap(s) in combination with the at least one single stranded overhang forms one or more unprocessed substrate moieties of said oligonucleotide probe,
d) oligonucleotide probes comprising unprocessed substrate moieties comprising one or more mismatched nucleobase hybridisation event(s) in the double stranded oligonucleotide probe,
e) oligonucleotide probes comprising unprocessed substrate moieties comprising the absence of nucleobase hybridisation(s) resulting in one or more loop formation(s) in the double stranded oligonucleotide probe, and
f) oligonucleotide probes comprising unprocessed substrate moieties comprising one or more damaged nucleotide(s) in the double stranded oligonucleotide probe,
or
a microfluidic device comprising one or more reaction compartments for performing one or more rolling circle amplification events of a circular oligonucleotide template and one or more detection compartments for the detection of said rolling circle amplification events performed in said one or more reaction compartments,
wherein a predetermined number of rolling circle amplification events correlate with a predetermined enzyme activity, and wherein the actual number of rolling circle amplification events recorded for a given sample is compared to the number of events correlating with said predetermined enzyme activity, thereby correlating the actual number of rolling circle amplification events with said activity of said one or more enzyme activities present in said sample.

180. A method for testing the efficacy of a drug or drug-lead, said method comprising the steps of
i) providing a drug or drug-lead to be tested;
ii) providing a biological sample to be treated with the drug or drug-lead;
iii) performing the correlation method of claim 179 for the biological sample in the absence of drug or drug-lead and determining the activity of one or more enzyme activities involved in circularising a non-circular oligonucleotide probe;
iv) contacting the drug or drug-lead and the biological sample;
v) performing the correlation method of claim 179 for the biological sample in the presence of drug or drug-lead and determining the activity of one or more enzyme activities involved in circularising a non-circular oligonucleotide probe;
vi) comparing the enzyme activities in the biological sample in the presence and absence, respectively, of the drug or drug-lead, wherein said comparison is obtained by comparing the rolling circle amplification events in the presence and absence, respectively, of the drug or drug-lead, and
vii) evaluating the efficacy of the drug or drug-lead based on the comparison performed in step vi).

181. A method for diagnosing or prognosing a disease in an individual by determining the activity of one or more enzyme activities involved in circularising a non-circular oligonucleotide probe, said method comprising the steps of
i) obtaining a biological sample from an individual to be tested, said biological sample comprising said one or more enzyme activities to be tested in the diagnostic or prognostic method,
ii) performing on said biological sample the method according to claim 1 and amplifying by rolling circle amplification the one or more circular templates having been generated as a result of the presence in said sample of said one or more enzyme activities being tested for, and optionally detecting said amplification events by using
a solid support comprising a plurality of attachment points for the attachment to the solid support of one or more oligonucleotide probes each comprising one or more unprocessed substrate moieties, wherein an oligonucleotide probe is either directly attached to an attachment point through one strand of the oligonucleotide probe, wherein said strand is capable of initiating rolling circle amplification of a second strand of the oligonucleotide probe, or an oligonucleotide probe is attached to an attachment point through hybridisation of the oligonucleotide probe to a primer oligonucleotide attached to an attachment point, wherein said primer is capable of initiating rolling circle amplification of the oligonucleotide probe, so that individual attachment points are associated with one or more oligonucleotide primers suitable for initiating rolling circle amplification of a circular template generated by enzyme processing of said one or more oligonucleotide probes each comprising one or more unprocessed substrate moieties, wherein the same or different primers are associated with the same or different attachment points, wherein the oligonucleotide probes attached to the solid support are selected from the group consisting of
a) oligonucleotide probes comprising unprocessed substrate moieties comprising one or more nick(s) in one or more single strand(s) of a double stranded nucleotide sequence of said oligonucleotide probe, said one or more nick(s) forming one or more unprocessed substrate moieties of said oligonucleotide probe,
b) oligonucleotide probes comprising unprocessed substrate moieties comprising one or more single stranded nucleotide sequence(s) joined at one or both ends thereof by a double stranded nucleotide sequence, said single stranded sequence(s) creating one or more gap structure(s) forming one or more unprocessed substrate moieties of said oligonucleotide probe,
c) oligonucleotide probes comprising unprocessed substrate moieties comprising one or more nick(s) or one or more gap(s), said gap(s) being in the form of a single stranded nucleotide sequence, said nick(s) or gap(s) being joined at one end thereof to a double stranded nucleotide sequence and at the other end thereof to at least one single stranded overhang joined to a double stranded nucleotide sequence of said oligonucleotide probe, wherein said nick(s) or gap(s) in combination with the at least one single stranded overhang forms one or more unprocessed substrate moieties of said oligonucleotide probe,
d) oligonucleotide probes comprising unprocessed substrate moieties comprising one or more mismatched nucleobase hybridisation event(s) in the double stranded oligonucleotide probe,
e) oligonucleotide probes comprising unprocessed substrate moieties comprising the absence of nucleobase hybridisation(s) resulting in one or more loop formation(s) in the double stranded oligonucleotide probe, and
f) oligonucleotide probes comprising unprocessed substrate moieties comprising one or more damaged nucleotide(s) in the double stranded oligonucleotide probe,
or
a microfluidic device comprising one or more reaction compartments for performing one or more rolling circle amplification events of a circular oligonucleotide template and one or more detection compartments for the detection of said rolling circle amplification events performed in said one or more reaction compartments, and
iii) determining the number of rolling circle amplification events and
iv) correlating said number of rolling amplification events with a predetermined enzyme activity corresponding to standard defining a physiologically normal activity of the one or more enzyme activities being tested for in a healthy individual,
wherein the actual number of rolling circle amplification events recorded for a given sample is compared to the number of events correlating with said predetermined enzyme activity, thereby correlating the actual number of rolling circle amplification events with said activity of said one or more enzyme activities present in said sample, and diagnosing or prognosing said individual with said disease, or the likelyhood of developing said disease, based on the enzyme activities determined in said biological sample.

182. A method for treating a disease diagnosed according to the method of claim 181, said method comprising the steps of administering a pharmaceutical composition to said individual having being diagnosed with said disease, wherein said medicament is capable of treating said disease by curing the disease or ameliorating the disease.

183. A method for treating prophylactically a disease prognosed according to the method of claim 181, said method comprising the steps of administering a pharmaceutical composition to said individual having being prognosed with the likelihood of developing said disease, wherein said pharmaceutical composition is capable of treating prophylactically said disease.

184-188. (canceled)

189. The method of claim 103, wherein said label is a fluorescent molecule incorporated into the rolling circle amplification product by being present in the primer used for probe amplification and generation of the rolling circle amplification product, by being linked to a nucleotide incorporated into the rolling circle amplification product during the probe amplification process, or by being linked to a fluorescently labelled oligonucleotide hybridising to the rolling circle amplification product.

190. The method of claim 103, wherein said label is a molecule or a chemical group which can be detected by a fluorescently labelled antibody.
